(19) United States
(12) Patent Application Publication
(10) Pub. No.: US 2022/0409679 A1
(43) Pub. Date: Dec. 29, 2022

(54) COMPOSITIONS AND METHODS WITH A PROBIOTIC AND A NUTRIENT AND/OR MINERAL FOR THE PREVENTION OR TREATMENT OF MASTITIS

(71) Applicant: SOCIETE DES PRODUITS NESTLE S.A., Vevey (CH)

(72) Inventor: TINU MARY SAMUEL, Ithaca, NY (US)

(21) Appl. No.: 17/756,570

(22) PCT Filed: Nov. 27, 2020
(86) PCT No.: PCT/EP2020/083623

§ 371 Date: May 27, 2022

(30) Foreign Application Priority Data
Nov. 29, 2019 (EP) 19212560.7

(51) Int. Cl. A61K 35/747; A61K 31/015; A61K 31/375; A61K 31/519; A61K 31/51; A61K 31/525; A61K 31/197; A61K 31/4415; A61K 31/714; A61K 33/30; A61K 33/32; A61K 33/26; A61K 33/04; A61K 33/34; A61K 33/06; A61K 33/00; A61K 38/38; A61K 31/685; A61K 31/202; A61K 31/122; A61P 29/00

(52) U.S. Cl. CPC A61K 35/747; A61K 31/015; A61K 31/375; A61K 31/519; A61K 31/51; A61K 31/525; A61K 31/197; A61K 31/4415; A61K 31/714; A61K 33/30; A61K 33/32; A61K 33/26; A61K 33/04; A61K 33/34; A61K 33/06; A61K 33/00; A61K 38/38; A61K 31/685; A61K 31/202; A61K 31/122; A61P 29/00

(57) ABSTRACT

The invention provides a mix or a nutritional composition comprising a probiotic Lactobacillus Fermentum CECT-5716 and a nutrient selected from the group consisting of beta-carotene, fiber, Vitamin C, Folate, Vitamin B1, Vitamin B2, Vitamin B5, Vitamin B6, Vitamin B12 and Potassium, or combination of two of more thereof, for use in treating or preventing mastitis, especially subclinical mastitis, in a subject, preferably a pregnant or lactating woman.

### FIELD OF THE INVENTION

The present invention relates to compositions for use in treating or preventing mastitis, for example sub-clinical mastitis, in a subject. In particular, the invention relates to the use of a probiotic and certain nutrients or minerals in treating or preventing mastitis, in particular sub-clinical mastitis.

### BACKGROUND TO THE INVENTION

WHO recommends that infants should be exclusively breastfed for the first six months of life to achieve optimal growth, development and health and continued breast feeding until 2 years of age. According to WHO, exclusive breastfeeding means that the infant receives only breast milk (no other liquids or solids are given—not even water—with the exception of oral rehydration solution, or drops/syrups of vitamins, minerals or medicines). WHO also recommends early initiation of breastfeeding as this may is critical to newborn survival and to establishing breastfeeding over the long term.

Mastitis is an inflammation of the mammary gland tissue, which can be classified as sub-clinical or clinical depending on the degree of inflammation.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain.

Staphylococcus infections, in particular S. aureus and S. epidermidis infections, are understood to be a primary cause of mastitis.

Mastitis can result in curtailment or even lack of initiation of breast-feeding of an infant.

Furthermore, the composition of breast milk may change during mastitis, for example increasing in content of sodium and inflammatory mediators, which may adversely affect the nutrition provided to the infant.

Current treatment of mastitis typically involves the administration of antibiotics. However, wide-spread use of antibiotics presents several challenges, including ineffectiveness due to antibiotic resistance, the creation of multiple-antibiotic resistant strains of bacteria, the formation of biofilms, vaginal candidiasis and antibiotic-associated diarrhoea.

Moreover, it has been indicated that there is insufficient evidence to support the effectiveness of antibiotic therapy for the treatment of lactational mastitis (Jahanfar, S. et al. (2013) Cochrane Database Syst Rev 28: CD005458).

Accordingly, there is a significant need for improved methods of treating and preventing mastitis.

There is a need to identify and provide solutions for preventing and treating mastitis in a “low-impact way”, especially in populations that can be fragile or not enable to tolerance drug intervention such as pregnant or lactating women

### SUMMARY OF THE INVENTION

The inventors have surprisingly found that a number of nutrients which are abundant in the diet of a group of lactating women were associated with a decreased occurrence of subclinical mastitis.

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

Another study suggests that a normal drop in [Na+] is highly predictive of successful lactation, although a prolonged elevation of [Na+] signifies impaired lactogenesis with a high risk of failure.

The inventors have studied the dietary intake of women with Na:K ratios greater than 0.6 and compared this to the dietary intake of normal women. Differences have been found in terms of median intake of certain nutrients, namely beta-carotene, fiber, Vitamin C, Folate, Vitamin B1, Vitamin B2, Vitamin B5, Vitamin B6, Vitamin B12 and Potassium.

Specifically, the inventors have found that women with sub-clinical mastitis have lower dietary intake of the following nutrients in comparison to normal women: beta-carotene, fiber, Vitamin C, Folate, Vitamin B1, Vitamin B2, Vitamin B5, Vitamin B6, Vitamin B12 or Potassium.

Supplementation with one or more of the above mentioned nutrients together with certain probiotics may therefore prevent or treat the sub-clinical mastitis.

Accordingly, in one aspect the invention provides a probiotic, preferably L. fermentum CECT-5716, together with a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof, for use in treating or preventing mastitis in a subject, for example sub-clinical mastitis.

In another aspect, the invention provides a a probiotic, preferably L. fermentum CECT-5716, together with beta-carotene for use in treating or preventing mastitis in a subject, for example sub-clinical mastitis, preferably wherein the beta-carotene is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides fiber together with a probiotic, preferably L. fermentum CECT-5716, for use in treating or preventing mastitis in a subject, for example sub-clinical mastitis, preferably wherein the fiber is in a combination with one or more nutrients selected in the group consisting of: beta-carotene, Vitamin C, Folate, Vitamin B1, Vitamin B2, Vitamin B5, Vitamin B6, Vitamin B12 and Potassium.

In another aspect, the invention provides Vitamin C with a probiotic, preferably L. fermentum CECT-5716, for use in treating or preventing mastitis in a subject, for example sub-clinical mastitis, preferably wherein the Vitamin C is in a combination with one or more nutrients selected in the group consisting of: beta-carotene, fiber, Folate, Vitamin B1, Vitamin B2, Vitamin B5, Vitamin B6, Vitamin B12 and Potassium.

In another aspect, the invention provides Folate together with a probiotic, preferably L. fermentum CECT-5716, for use in treating or preventing mastitis in a subject, for example sub-clinical mastitis, preferably wherein the Folate is in a combination with one or more nutrients selected in the group consisting of: beta-carotene, fiber, Vitamin C, Vitamin B1, Vitamin B2, Vitamin B5, Vitamin B6, Vitamin B12 and Potassium.

In another aspect, the invention provides Potassium together with a probiotic, preferably L. fermentum CECT-5716, for use in treating or preventing mastitis in a subject, for example sub-clinical mastitis, preferably wherein the Potassium is in a combination with one or more nutrients selected in the group consisting of: beta-carotene, fiber, Vitamin C, Vitamin B1, Vitamin B2, Vitamin B5, Vitamin B6, Vitamin B12 and Folate.

In another aspect, the invention provides Vitamin B1 together with a probiotic, preferably L. fermentum CECT-5716, for use in treating or preventing mastitis in a subject, for example sub-clinical mastitis, preferably wherein the vitamin B1 is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides Vitamin B2 together with a probiotic, preferably L. fermentum CECT-5716, for use in treating or preventing mastitis in a subject, for example sub-clinical mastitis, preferably wherein the vitamin B2 is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides Vitamin B5 together with a probiotic, preferably L. fermentum CECT-5716, for use in treating or preventing mastitis in a subject, for example sub-clinical mastitis, preferably wherein the Vitamin B5 is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides Vitamin B6 together with a probiotic, preferably L. fermentum CECT-5716, for use in treating or preventing mastitis in a subject, for example sub-clinical mastitis, preferably wherein the Vitamin B6 is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides Vitamin B12 together with a probiotic, preferably L. fermentum CECT-5716, for use in treating or preventing mastitis in a subject, for example sub-clinical mastitis, preferably wherein the Vitamin B12 is in a combination with one or more nutrients selected in the group consisting of: beta-carotene, fiber, Vitamin C, Folate, Vitamin B1, Vitamin B2, Vitamin B5, Vitamin B6, Vitamin B12 and Potassium.

In another aspect, the invention provides beta-carotene together with a probiotic, preferably L. fermentum CECT-5716, for use in treating or preventing mastitis in a subject, for example sub-clinical mastitis, wherein the beta-carotene is administered to the subject in combination with one or more nutrients selected in the group consisting of: fiber, Vitamin C, Folate, Vitamin B1, Vitamin B2, Vitamin B5, Vitamin B6, Vitamin B12 and Potassium. In one embodiment, the beta-carotene is administered to the subject simultaneously, sequentially or separately in combination with one or more nutrients selected in the group consisting of: fiber, Vitamin C, Folate, Vitamin B1, Vitamin B2, Vitamin B5, Vitamin B6, Vitamin B12 and Potassium, preferably simultaneously.

In another aspect, the invention provides fiber together with a probiotic, preferably L. Fermentum CECT-5716, for use in treating or preventing mastitis in a subject, for example sub-clinical mastitis, wherein the fiber is administered to the subject in combination with one or more nutrients selected in the group consisting of: beta-carotene, Vitamin C, Folate, Vitamin B1, Vitamin B2, Vitamin B5, Vitamin B6, Vitamin B12 and Potassium. In one embodiment, the fiber is administered to the subject simultaneously, sequentially or separately in combination with one or more nutrients selected in the group consisting of: beta-carotene, Vitamin C, Folate, Vitamin B1, Vitamin B2, Vitamin B5, Vitamin B6, Vitamin B12 and Potassium, preferably simultaneously.

In another aspect, the invention provides Vitamin C together with a probiotic, preferably L. fermentum CECT-5716, for use in treating or preventing mastitis in a subject, for example sub-clinical mastitis, wherein the Vitamin C is administered to the subject in combination with one or more nutrients selected in the group consisting of: beta-carotene, fiber, Folate, Vitamin B1, Vitamin B2, Vitamin B5, Vitamin B6, Vitamin B12 and Potassium. In one embodiment, the Vitamin C is administered to the subject simultaneously, sequentially or separately in combination with one or more nutrients selected in the group consisting of: beta-carotene, fiber, Folate, Vitamin B1, Vitamin B2, Vitamin B5, Vitamin B6, Vitamin B12 and Potassium, preferably simultaneously.

In another aspect, the invention provides Folate together with a probiotic, preferably L. fermentum CECT-5716, for use in treating or preventing mastitis in a subject, for example sub-clinical mastitis, wherein the Folate is administered to the subject in combination with one or more nutrients selected in the group consisting of: beta-carotene, fiber, Vitamin C, Vitamin B1, Vitamin B2, Vitamin B5, Vitamin B6, Vitamin B12 and Potassium. In one embodiment, the Folate is administered to the subject simultaneously, sequentially or separately in combination with one or more nutrients selected in the group consisting of: beta-carotene, fiber, Vitamin C, Vitamin B1, Vitamin B2, Vitamin B5, Vitamin B6, Vitamin B12 and Potassium, preferably simultaneously.

In another aspect, the invention provides Potassium together with a probiotic, preferably L. fermentum CECT-5716, for use in treating or preventing mastitis in a subject, for example sub-clinical mastitis, wherein the Potassium is administered to the subject in combination with one or more nutrients selected in the group consisting of: beta-carotene, fiber, Vitamin C, Vitamin B1, Vitamin B2, Vitamin B5, Vitamin B6, Vitamin B12 and Folate. In one embodiment, the Potassium is administered to the subject simultaneously, sequentially or separately in combination with one or more nutrients selected in the group consisting of: beta-carotene, fiber, Vitamin C, Vitamin B1, Vitamin B2, Vitamin B5, Vitamin B6, Vitamin B12 and Folate, preferably simultaneously.

In another aspect, the invention provides Vitamin B1 together with a probiotic, preferably L. fermentum CECT-5716, for use in treating or preventing mastitis in a subject, for example sub-clinical mastitis, wherein the Vitamin B1 is administered to the subject in combination with one or more nutrients selected in the group consisting of: beta-carotene, fiber, Vitamin C, Folate, Vitamin B2, Vitamin B5, Vitamin B6, Vitamin B12 and Potassium. In one embodiment, the Vitamin B1 is administered to the subject simultaneously, sequentially or separately in combination with one or more nutrients selected in the group consisting of: beta-carotene, fiber, Vitamin C, Folate, Vitamin B2, Vitamin B5, Vitamin B6, Vitamin B12 and Potassium, preferably simultaneously.

In another aspect, the invention provides Vitamin B2 together with a probiotic, preferably L. fermentum CECT-5716, for use in treating or preventing mastitis in a subject, for example sub-clinical mastitis, wherein the Vitamin B2 is administered to the subject in combination with one or more nutrients selected in the group consisting of: beta-carotene, fiber, Vitamin C, Folate, Vitamin B1, Vitamin B5, Vitamin B6, Vitamin B12 and Potassium. In one embodiment, the Vitamin B2 is administered to the subject simultaneously, sequentially or separately in combination with one or more nutrients selected in the group consisting of: beta-carotene, fiber, Vitamin C, Folate, Vitamin B1, Vitamin B5, Vitamin B6, Vitamin B12 and Potassium, preferably simultaneously.

In another aspect, the invention provides Vitamin B5 together with a probiotic, preferably L. fermentum CECT-5716, for use in treating or preventing mastitis in a subject, for example sub-clinical mastitis, wherein the Vitamin B5 is administered to the subject in combination with one or more nutrients selected in the group consisting of: beta-carotene, fiber, Vitamin C, Folate, Vitamin B1, Vitamin B2, Vitamin B6, Vitamin B12 and Potassium. In one embodiment, the Vitamin B5 is administered to the subject simultaneously, sequentially or separately in combination with one or more nutrients selected in the group consisting of: beta-carotene, fiber, Vitamin C, Folate, Vitamin B1, Vitamin B2, Vitamin B6, Vitamin B12 and Potassium, preferably simultaneously.

In another aspect, the invention provides Vitamin B6 together with a probiotic, preferably L. fermentum CECT-5716, for use in treating or preventing mastitis in a subject, for example sub-clinical mastitis, wherein the Vitamin B6 is administered to the subject in combination with one or more nutrients selected in the group consisting of: beta-carotene, fiber, Vitamin C, Folate, Vitamin B1, Vitamin B2, Vitamin B5, Vitamin B12 and Potassium. In one embodiment, the Vitamin B6 is administered to the subject simultaneously, sequentially or separately in combination with one or more nutrients selected in the group consisting of: beta-carotene, fiber, Vitamin C, Folate, Vitamin B1, Vitamin B2, Vitamin B5, Vitamin B12 and Potassium, preferably simultaneously.

In another aspect, the invention provides Vitamin B12 together with a probiotic, preferably L. fermentum CECT-5716, for use in treating or preventing mastitis in a subject, for example sub-clinical mastitis, wherein the Vitamin B12 is administered to the subject in combination with one or more nutrients selected in the group consisting of: beta-carotene, fiber, Vitamin C, Folate, Vitamin B1, Vitamin B2, Vitamin B5, Vitamin B6 and Potassium. In one embodiment, the Vitamin B12 together with a probiotic, preferably L. fermentum CECT-5716, is administered to the subject simultaneously, sequentially or separately in combination with one or more nutrients selected in the group consisting of: beta-carotene, fiber, Vitamin C, Folate, Vitamin B1, Vitamin B2, Vitamin B5, Vitamin B6 and Potassium, preferably simultaneously.

In another aspect, the invention provides a combination of two or more nutrients (together with a probiotic, preferably L. fermentum CECT-5716), selected from the group consisting of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 for use in treating or preventing mastitis in a subject, for example sub-clinical mastitis.

In one embodiment, two or more of (a), (b), (c), (d) (e), (f), (g), (h), (i) and (j) are administered to the subject simultaneously, sequentially or separately.

In a preferred embodiment, two or more of (a), (b), (c), (d) (e), (f), (g), (h), (i) and (j) are administered to the subject simultaneously.

In another aspect, the invention provides a composition comprising one or more nutrients (together with a probiotic, preferably L. fermentum CECT-5716), selected from the group consisting of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 for use in treating or preventing mastitis in a subject, for example sub-clinical mastitis.

In another aspect, the invention provides a method for treating or preventing mastitis, for example sub-clinical mastitis, wherein the method comprises administering one or more nutrients (together with a probiotic, preferably L. fermentum CECT-5716), selected from the group consisting of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 to a subject in need thereof.

In another aspect, the invention provides a combination of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12, together with a probiotic, preferably L. fermentum CECT-5716, for use in treating or preventing mastitis in a subject, for example sub-clinical mastitis, preferably wherein (a)-(i) are administered to the subject simultaneously, sequentially or separately, more preferably wherein (a)-(i) are administered to the subject simultaneously.

In another aspect, the invention provides a composition comprising (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12, together with a probiotic, preferably L. fermentum CECT-5716, for use in treating or preventing mastitis in a subject, for example sub-clinical mastitis.

In another aspect, the invention provides a method for treating or preventing mastitis, for example sub-clinical mastitis, wherein the method comprises administering (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12, together with a probiotic, preferably L. fermentum CECT-5716, to a subject in need thereof, preferably wherein the probiotic, the (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 are administered to the subject simultaneously, sequentially or separately, more preferably wherein the (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 are administered to the subject simultaneously.

In another aspect, the invention provides a nutrient selected from the group consisting of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 and a combination of two of more thereof, together with a probiotic, preferably L. fermentum CECT-5716, for use in reducing the risk of mastitis in a subject, for example sub-clinical mastitis.

In another aspect, the invention provides a combination of two or more nutrients, (together with a probiotic, preferably L. fermentum CECT-5716), selected from the group consisting of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 for use in reducing the risk of mastitis in a subject, for example sub-clinical mastitis.

In another aspect, the invention provides beta-carotene together with a probiotic, preferably L. fermentum CECT-5716, for use in reducing the risk of mastitis in a subject, for example sub-clinical mastitis, preferably wherein the beta-carotene is in a combination with one or more nutrients selected in the group consisting of: beta-carotene, fiber, Vitamin C, Folate, Potassium, Vitamin B1, Vitamin B2, Vitamin B5, (Vitamin B6 and Vitamin B12.

In another aspect, the invention provides fiber together with a probiotic, preferably L. fermentum CECT-5716, for use in reducing the risk of mastitis in a subject, for example sub-clinical mastitis, preferably wherein the fiber is in a combination with one or more nutrients selected in the group consisting of: beta-carotene, Vitamin C, Folate, Potassium, Vitamin B1, Vitamin B2, Vitamin B5, Vitamin B6 and Vitamin B12.

In another aspect, the invention provides Vitamin C together with a probiotic, preferably L. fermentum CECT-5716, for use in reducing the risk of mastitis in a subject, for example sub-clinical mastitis, preferably wherein the Vitamin C is in a combination with one or more nutrients selected in the group consisting of: beta-carotene, fiber, Folate, Vitamin B1, Vitamin B2, Vitamin B5, Vitamin B6, Vitamin B12, and Potassium.

In another aspect, the invention provides Folate together with a probiotic, preferably L. fermentum CECT-5716, for use in reducing the risk of mastitis in a subject, for example sub-clinical mastitis, preferably wherein the Folate is in a combination with one or more nutrients selected in the group consisting of: beta-carotene, fiber, Vitamin C, Vitamin B1, Vitamin B2, Vitamin B5, Vitamin B6, Vitamin B12 and Potassium.

In another aspect, the invention provides Potassium, together with a probiotic, preferably L. fermentum CECT-5716, for use in reducing the risk of mastitis in a subject, for example sub-clinical mastitis, preferably wherein the Potassium is in a combination with one or more nutrients selected in the group consisting of: beta-carotene, fiber, Vitamin C, Vitamin B1, Vitamin B2, Vitamin B5, Vitamin B6, Vitamin B12 and Folate.

In another aspect, the invention provides Vitamin B1, together with a probiotic, preferably L. fermentum CECT-5716, for use in reducing the risk of mastitis in a subject, for example sub-clinical mastitis, preferably wherein the Vitamin B1 is in a combination with one or more nutrients selected in the group consisting of: beta-carotene, fiber, Vitamin C, Folate, Potassium, Vitamin B1, Vitamin B2, Vitamin B5, Vitamin B6 and Vitamin B12.

In another aspect, the invention provides Vitamin B2, together with a probiotic, preferably L. fermentum CECT-5716, for use in reducing the risk of mastitis in a subject, for example sub-clinical mastitis, preferably wherein the Vitamin B2 is in a combination with one or more nutrients selected in the group consisting of: beta-carotene, fiber, Vitamin C, Folate, Potassium, Vitamin B1, Vitamin B2, Vitamin B5, Vitamin B6 and Vitamin B12.

In another aspect, the invention provides Vitamin B5, together with a probiotic, preferably L. fermentum CECT-5716, for use in reducing the risk of mastitis in a subject, for example sub-clinical mastitis, preferably wherein the Vitamin B5 is in a combination with one or more nutrients selected in the group consisting of: beta-carotene, fiber, Vitamin C, Folate, Potassium, Vitamin B1, Vitamin B2, Vitamin B5, Vitamin B6 and Vitamin B12.

In another aspect, the invention provides Vitamin B6, together with a probiotic, preferably L. fermentum CECT-5716, for use in reducing the risk of mastitis in a subject, for example sub-clinical mastitis, preferably wherein the Vitamin B6 is in a combination with one or more nutrients selected in the group consisting of: beta-carotene, fiber, Vitamin C, Folate, Potassium, Vitamin B1, Vitamin B2, Vitamin B5, Vitamin B6 and Vitamin B12.

In another aspect, the invention provides Vitamin B12, together with a probiotic, preferably L. fermentum CECT-5716, for use in reducing the risk of mastitis in a subject, for example sub-clinical mastitis, preferably wherein the Vitamin B12 is in a combination with one or more nutrients selected in the group consisting of: beta-carotene, fiber, Vitamin C, Folate, Potassium, Vitamin B1, Vitamin B2, Vitamin B5, Vitamin B6 and Vitamin B12.

In another aspect, the invention provides beta-carotene, together with a probiotic, preferably L. fermentum CECT-5716, for use in reducing the risk of mastitis in a subject, for example sub-clinical mastitis, wherein the beta-carotene is administered to the subject in combination with one or more nutrients selected in the group consisting of: fiber, Vitamin C, Folate, Vitamin B1, Vitamin B2, Vitamin B5, Vitamin B6, Vitamin B12 and Potassium. In one embodiment, the beta-carotene is administered to the subject simultaneously, sequentially or separately in combination with one or more nutrients selected in the group consisting of: fiber, Vitamin C, Folate, Vitamin B1, Vitamin B2, Vitamin B5, Vitamin B6, Vitamin B12 and Potassium, preferably simultaneously.

In another aspect, the invention provides fiber together with a probiotic, preferably L. fermentum CECT-5716, for use in reducing the risk of mastitis in a subject, for example sub-clinical mastitis, wherein the fiber is administered to the subject in combination with one or more nutrients selected in the group consisting of: beta-carotene, Vitamin C, Folate, Vitamin B1, Vitamin B2, Vitamin B5, Vitamin B6, Vitamin B12 and Potassium. In one embodiment, the fiber is administered to the subject simultaneously, sequentially or separately in combination with one or more nutrients selected in the group consisting of: beta-carotene, Vitamin C, Folate, Vitamin B1, Vitamin B2, Vitamin B5, Vitamin B6, Vitamin B12 and Potassium, preferably simultaneously.

In another aspect, the invention provides Vitamin C together with a probiotic, preferably L. fermentum CECT-5716, for use in reducing the risk of mastitis in a subject, for example sub-clinical mastitis, wherein the Vitamin C is administered to the subject in combination with one or more nutrients selected in the group consisting of: beta-carotene, fiber, Folate, Vitamin B1, Vitamin B2, Vitamin B5, Vitamin B6, Vitamin B12 and Potassium. In one embodiment, the Vitamin C is administered to the subject simultaneously, sequentially or separately in combination with one or more nutrients selected in the group consisting of: beta-carotene, fiber, Folate, Potassium, Vitamin B1, Vitamin B2, Vitamin B5, (Vitamin B6 and Vitamin B12, preferably simultaneously.

In another aspect, the invention provides Folate together with a probiotic, preferably L. fermentum CECT-5716, for use in reducing the risk of mastitis in a subject, for example sub-clinical mastitis, wherein the Folate is administered to the subject in combination with one or more nutrients selected in the group consisting of: beta-carotene, fiber, Vitamin C, Vitamin B1, Vitamin B2, Vitamin B5, Vitamin B6, Vitamin B12 and Potassium. In one embodiment, the Folate is administered to the subject simultaneously, sequentially or separately in combination with one or more nutrients selected in the group consisting of: beta-carotene, fiber, Vitamin C, Vitamin B1, Vitamin B2, Vitamin B5, Vitamin B6, Vitamin B12 and Potassium, preferably simultaneously.

In another aspect, the invention provides Potassium, together with a probiotic, preferably L. fermentum CECT-5716, for use in reducing the risk of mastitis in a subject, for example sub-clinical mastitis, wherein the Potassium is administered to the subject in combination with one or more nutrients selected in the group consisting of: beta-carotene, fiber, Vitamin C, Vitamin B1, Vitamin B2, Vitamin B5, Vitamin B6, Vitamin B12 and Folate. In one embodiment, the Potassium is administered to the subject simultaneously, sequentially or separately in combination with one or more nutrients selected in the group consisting of: beta-carotene, fiber, Vitamin C, Vitamin B1, Vitamin B2, Vitamin B5, Vitamin B6, Vitamin B12 and Folate, preferably simultaneously.

In another aspect, the invention provides Vitamin B1, together with a probiotic, preferably L. fermentum CECT-5716, for use in reducing the risk of mastitis in a subject, for example sub-clinical mastitis, wherein the Vitamin B1 is administered to the subject in combination with one or more nutrients selected in the group consisting of: fiber, Vitamin C, Folate, beta-carotene, Vitamin B2, Vitamin B5, Vitamin B6, Vitamin B12 and Potassium. In one embodiment, the Vitamin B1 is administered to the subject simultaneously, sequentially or separately in combination with one or more nutrients selected in the group consisting of: fiber, Vitamin C, Folate, beta-carotene, Vitamin B2, Vitamin B5, Vitamin B6, Vitamin B12 and Potassium, preferably simultaneously.

In another aspect, the invention provides Vitamin B2, together with a probiotic, preferably L. fermentum CECT-5716, for use in reducing the risk of mastitis in a subject, for example sub-clinical mastitis, wherein the Vitamin B2 is administered to the subject in combination with one or more nutrients selected in the group consisting of: fiber, Vitamin C, Folate, beta-carotene, Vitamin B1, Vitamin B5, Vitamin B6, Vitamin B12 and Potassium. In one embodiment, the Vitamin B2 is administered to the subject simultaneously, sequentially or separately in combination with one or more nutrients selected in the group consisting of: fiber, Vitamin C, Folate, beta-carotene, Vitamin B2, Vitamin B5, Vitamin B6, Vitamin B12 and Potassium, preferably simultaneously.

In another aspect, the invention provides Vitamin B5, together with a probiotic, preferably L. fermentum CECT-5716, for use in reducing the risk of mastitis in a subject, for example sub-clinical mastitis, wherein the Vitamin B5 is administered to the subject in combination with one or more nutrients selected in the group consisting of: fiber, Vitamin C, Folate, beta-carotene, Vitamin B1, Vitamin B2, Vitamin B6, Vitamin B12 and Potassium. In one embodiment, the Vitamin B5 is administered to the subject simultaneously, sequentially or separately in combination with one or more nutrients selected in the group consisting of: fiber, Vitamin C, Folate, beta-carotene, Vitamin B2, Vitamin B1, Vitamin B6, Vitamin B12 and Potassium, preferably simultaneously.

In another aspect, the invention provides Vitamin B6, together with a probiotic, preferably L. fermentum CECT-5716, for use in reducing the risk of mastitis in a subject, for example sub-clinical mastitis, wherein the Vitamin B6 is administered to the subject in combination with one or more nutrients selected in the group consisting of: fiber, Vitamin C, Folate, beta-carotene, Vitamin B1, Vitamin B2, Vitamin B5, Vitamin B12 and Potassium. In one embodiment, the Vitamin B6 is administered to the subject simultaneously, sequentially or separately in combination with one or more nutrients selected in the group consisting of: fiber, Vitamin C, Folate, beta-carotene, Vitamin B2, Vitamin B1, Vitamin B5, Vitamin B12 and Potassium, preferably simultaneously.

In another aspect, the invention provides Vitamin B12, together with a probiotic, preferably L. fermentum CECT-5716, for use in reducing the risk of mastitis in a subject, for example sub-clinical mastitis, wherein the Vitamin B12 is administered to the subject in combination with one or more nutrients selected in the group consisting of: fiber, Vitamin C, Folate, beta-carotene, Vitamin B1, Vitamin B2, Vitamin B5, Vitamin B6 and Potassium. In one embodiment, the Vitamin B12 is administered to the subject simultaneously, sequentially or separately in combination with one or more nutrients selected in the group consisting of: fiber, Vitamin C, Folate, beta-carotene, Vitamin B2, Vitamin B1, Vitamin B5, Vitamin B6 and Potassium, preferably simultaneously.

In another aspect, the invention provides a composition comprising one or more nutrients selected from the group consisting of beta-carotene, fiber, Vitamin C, Folate, Vitamin B1, Vitamin B2, Vitamin B5, Vitamin B6, Vitamin B12 and Potassium, together with a probiotic, preferably L. fermentum CECT-5716, for use in reducing the risk of mastitis in a subject, for example sub-clinical mastitis.

In another aspect, the invention provides a method for reducing the risk of mastitis, for example sub-clinical mastitis, wherein the method comprises administering one or more nutrients selected from the group consisting of beta-carotene, fiber, Vitamin C, Folate, Vitamin B1, Vitamin B2, Vitamin B5, Vitamin B6, Vitamin B12 and Potassium, together with a probiotic, preferably L. fermentum CECT-5716, to a subject in need thereof.

In another aspect, the invention provides a combination of (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12, together with a probiotic, preferably L. Fermentum CECT-5716, for use in reducing the risk of mastitis in a subject, for example sub-clinical mastitis.

In another aspect, the invention provides a composition comprising (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12, together with a probiotic, preferably L. Fermentum CECT-5716, for use in reducing the risk of mastitis in a subject, for example sub-clinical mastitis.

In another aspect, the invention provides a method for reducing the risk of mastitis, for example sub-clinical mastitis, wherein the method comprises administering a probiotic, preferably L. fermentum CECT-5716, (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 to a subject in need thereof, preferably wherein the (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 are administered to the subject simultaneously, sequentially or separately, more preferably wherein the (a) beta-carotene, (b) fiber, (c) Vitamin C, (d) Folate, (e) Potassium, (f) Vitamin B1, (g) Vitamin B2, (h) Vitamin B5, (i) Vitamin B6 and (j) Vitamin B12 are administered to the subject simultaneously.

### DETAILED DESCRIPTION OF THE INVENTION

### Definitions

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Within the context of the present invention, the term “nutrient” or nutrients” is intended to comprise both macronutrients (for example carbohydrates, proteins or fats) and micronutrients (for example minerals or vitamins) for the human body.

Within the context of the present invention, the term “ingredient” or “ingredients” indicates an edible substance or mixture of substances which comprise or is essentially consisting of a nutrient for the human body.

In one embodiment of the present invention, the term “ingredient” or “ingredients” indicates an edible substance essentially consisting of a nutrient for the human body.

Within the context of the present invention, the term “ingredient providing nutrient X” or “ingredients providing nutrient X” indicates an edible substance and/or mixture of substances which comprise or is essentially consisting of at least one substance capable of delivering the specified nutrient X to the human body.

Within the context of the present invention, the term “ingredient providing nutrient X in amount Y” or “ingredients providing nutrient X in amount Y” indicates an edible substance and/or mixture of substances which comprise or is essentially consisting of at least one substance capable of delivering the specified nutrient X to the human body in the specified amount Y.

Insoluble fiber does not dissolve in water, is metabolically inert and provides bulking, or it can be prebiotic and metabolically ferment in the large intestine. Chemically, dietary fiber consists of non-starch polysaccharides (NPS) such as arabinoxylans, cellulose, and many other plant components such as resistant oligosaccharides, resistant starch, resistant dextrins, inulin, lignin, chitins, pectins, beta-glucans, and oligosaccharides. Non limiting examples of dietary fibers are: prebiotic fibers such as Fructo-oligosaccharides (FOS), inulin, galacto-oligosaccharides (GOS), fruit fiber, legume fiber, vegetable fiber, cereal fiber, resistant starch such as high amylose corn starch. As fibers are not digestible, they do not contain available carbohydrates.

The expressions “fiber” or “fibers” or “dietary fiber” or “dietary fibers” within the context of the present invention indicate the indigestible portion, in small intestine, of food derived from plants which comprises two main components: Soluble fiber, which dissolves in water, and insoluble fiber. Mixtures of fibers are comprised within the scope of the terms above mentioned. Soluble fiber is readily fermented in the colon into gases and physiologically active byproducts, and can be prebiotic and viscous.

Within the context of the present invention the term “added fiber” or “added dietary fiber” indicates an ingredient mainly or totally constituted by fiber which is added to the composition according to the present invention and whose content in fiber contributes to the total fiber content of the composition. The total fiber content of the composition is provided by the sum of amount of fiber naturally present in ingredients used in the recipe (for example from whole grain cereal flour) plus amount of added fiber.

Within the context of the present invention, the term “legume” or “legumes” identifies the fruit or seed of a plant in the family of Fabaceae or mixtures thereof. Well-known legumes include inter alia alfalfa, clover, peas, beans, lentils, lupins, mesquite, carob, soybeans, peanuts and tamarind. The grain seeds of such plants are generally known as “pulses” and are comprised within the scope of the term “legumes” according to the present invention.

Within the context of the present invention, the term “fruit” or “fruits” indicates ingredients derived from fruit such as for example fresh fruit, fruit paste, dried fruit, fruit extracts and/or centrifugates. Mixtures of such ingredients are also comprised within the scope of the terms above mentioned. Non limiting examples of fruit according to the present invention are: apple, apricot, banana, cherry, pear, strawberry, Mango, Orange, peach.

As it will be apparent to the skilled person, legumes and fruit according to the present invention may bring certain amount of fibers to the composition of the present invention.

The composition of the present invention, including the many embodiments described herein, can comprise, consist of, or consist essentially of the essential elements and limitations of the invention described herein, as well as any additional or optional ingredients, components, or limitations described herein.

### EMBODIMENTS OF THE INVENTION

In one embodiment the present invention comprises a probiotic L. fermentum CECT-5716 together with a nutrient and/or a mineral

In one embodiment of the present invention, the composition comprises any mineral and/or vitamin in an amount of at least 15% of the recommended daily allowance (RDA).

In one embodiment, the mastitis is sub-clinical mastitis or clinical mastitis.

In a preferred embodiment, the mastitis is sub-clinical mastitis.

In one embodiment, the subject is at risk of suffering from sub-clinical mastitis or clinical mastitis.

In one embodiment, the risk of suffering from mastitis (such as sub-clinical mastitis or clinical mastitis) is indicated by the presence of one or more risk factors selected from the group consisting of family history of sub-clinical mastitis or clinical mastitis, breast-feeding attachment difficulties, mother-infant separation (e.g. separation of greater than 24 h), blocked duct, milk stasis, cracked nipples, pre-lacteal feeds, milk oversupply, breast engorgement, feeding from alternate breasts on consecutive feeds, infant mouth abnormalities, a short infant frenulum, maternal use of antibiotics, previous history of mastitis in the subject, maternal stress, delivery in private versus public hospital and the presence of Staphylococcus aureus in milk.

In one embodiment, the subject is a human e.g. a woman who is desiring to get pregnant, who is pregnant or who is lactating.

In one embodiment, the subject is a human e.g. a lactating woman. In another embodiment, the subject is a lactating woman.

In an additional embodiment, the subject is a European lactating woman.

In one embodiment, the subject is a livestock animal or a companion animal. In one embodiment, the subject is a cow or dog. In another embodiment, the subject is a rat or mouse.

In one embodiment, the treatment or prevention increases the probability, likelihood or chances of success of initiating and/or continuing and/or prolonging breastfeeding by the subject.

In one embodiment, the treatment or prevention increases the probability, likelihood or chances of success of the subject exclusively breast-feeding her infant.

In one embodiment, the treatment or prevention increases the duration (length of time e.g. number of days, weeks, months) of breastfeeding by the subject.

In one embodiment, the subject is able to breast-feed for at least 4 months, preferably 4-24 months, optionally 4-6 months.

In one embodiment, the subject is able to breast-feed for at least 6 months, preferably 6-24 months.

In one embodiment, the subject continues to breast-feed for at least 4 months, preferably 4-24 months, optionally 4-6 months.

In one embodiment, the subject continues to breast-feed for at least 6 months, preferably 6-24 months.

In one embodiment, the treatment or prevention increases the quality of the subject's breast milk. In one embodiment, the treatment or prevention increases the vitamers and/or biomarkers related to the administered nutrients in human breast milk.

In one embodiment, the treatment or preventing increases the quantity of the subject's breast milk.

In one embodiment, the composition is a nutritional composition or a pharmaceutical composition, preferably a nutritional composition.

In one embodiment, the composition is a maternal nutritional composition, preferably for use during lactation and/or pregnancy, for example late pregnancy. In another embodiment, the composition is a maternal nutritional composition for use during lactation.

In one embodiment, the nutrient or composition for use according to the present invention is in the form of a tablet, gel capsule, powder, maternal milk powder, food product, liquid format (e.g. ready to drink format) and/or beverage.

Fiber

In one embodiment of the present invention, a composition is provided which comprises at least one ingredient which delivers fibres.

In one embodiment of the present invention, a food composition is provided which comprises fibres.

In one embodiment, the ingredients providing fibres may be capable of providing fibres of natural or synthetic origin.

In one embodiment, fibres of synthetic origin are for example FOS from sucrose.

In one embodiment, ingredients which are capable of providing dietary fibres are selected in the group consisting of: Fruit, Vegetable, Legume, Cereal and Cruciferous vegetable.

In one embodiment, dietary fibres are selected in the group consisting of: resistant dextrin, resistant oligosaccharides, NPS, resistant starches (for example pectine), polydextrose, inulin, partially hydrolyzed guar gum (PHGG), FOS, acacia gum, pea fiber, and mixtures thereof.

As it is evident to the person skilled in the art, different ingredients may provide different amounts of dietary fibres in the composition according to the present invention, depending on the nature and amount of the ingredient used. It will be nonetheless routine work to the skilled person to calculate the amount of ingredient needed to provide the claimed amount of dietary fibres, based on the specification of the specific ingredient provided by the supplier.

In one embodiment, the dosage of fibers is at least 17 g/day. In a further embodiment, the dosage of fibers is at least 20 g/day. In a still further embodiment, the dosage of fibers is ranging from 16 to 30 g/day, for example 20 to 25 g/day.

In one embodiment, the composition according to the present invention comprises at least 17 g of fibers per serving. In a further embodiment, the composition according to the present invention comprises at least 20 g fibers per serving. In a still further embodiment, the composition comprises fibers in an amount ranging from 16 to 30 g, for example 20 to 25 g per serving.

In such embodiment, the composition of the present invention delivers the daily amount of fibers considered responsible for the beneficial observed effect on occurrence of mastitis, especially subclinical mastitis.

In one embodiment, the composition according to the present invention comprises at least 4 g of fibers. In a still further embodiment, the composition comprises fibers in an amount ranging from 2 to 30 g, for example 2 to 25 g.

In such embodiment, the composition of the present invention delivers the daily amount of fibers resulting to be missing in lactating women according to our study and necessary to reach levels associated with the beneficial observed effect on occurrence of mastitis, especially subclinical mastitis.

In one embodiment, the composition according to the present invention comprises at least 4 g of fibers. In a still further embodiment, the composition comprises fibers in an amount ranging from 4 to 30 g, for example 4 to 25 g.

In such embodiment, the composition of the present invention delivers the daily amount of fibers resulting to be missing in lactating women with subclinical mastitis according to our study and necessary to reach levels associated with the beneficial observed effect on occurrence of mastitis, especially subclinical mastitis.

In one embodiment, the dosage of fibers as above described is adapted for a lactating woman.

The fibers may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of fibers as above described. In such embodiment, as it will be apparent to a person skilled in the art, the amount of fibers contained in each serving of the composition according to the present invention will be divided by 1, 2 3 or 4 respectively.

In one embodiment, the composition according to the present invention is intended for consumption once or twice per day.

Beta-Carotene

Beta-carotene may be incorporated in the composition of the invention as such or via any source comprising it. For example ingredients providing beta-carotene may be synthetic or natural sources.

As it is evident to the person skilled in the art, different ingredients may provide different amounts of beta-carotene in the composition according to the present invention, depending on the nature and amount of the ingredient used. It will be nonetheless routine work to the skilled person to calculate the amount of ingredient needed to provide the claimed amount of beta-carotene, based on the specification of the specific ingredient provided by the supplier.

In one embodiment, the dosage of beta-carotene is at least 2100 μg/day. In a further embodiment, the dosage of beta-carotene is at least 2400 μg/day. In a still further embodiment, the dosage of beta-carotene is ranging from 2100 to 3500 μg/day, for example from 2400 to 3500 □g/day.

In one embodiment, the composition according to the present invention comprises at least 2100 μg of beta-carotene. In a further embodiment, the composition according to the present invention comprises at least 2400 μg beta-carotene. In a still further embodiment, the composition comprises beta carotene in an amount ranging from 2100 to 3500 μg, for example from 2400 to 3500 μg.

In such embodiment, the composition of the present invention delivers the daily amount of beta-carotene considered responsible for the beneficial observed effect on occurrence of mastitis, especially subclinical mastitis.

In one embodiment, the composition according to the present invention comprises at least 670 μg of beta-carotene. In a further embodiment, the composition according to the present invention comprises at least 800 μg beta-carotene. In a still further embodiment, the composition comprises beta carotene in an amount ranging from 670 to 3500 μg, for example from 800 to 3500 μg. In such embodiment, the composition of the present invention delivers the daily amount of beta carotene resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 340 μg of beta-carotene. In a further embodiment, the composition according to the present invention comprises at least 400 μg beta-carotene. In a still further embodiment, the composition comprises beta carotene in an amount ranging from 340 to 3500 μg, for example from 400 to 3500 μg.

In such embodiment, the composition of the present invention delivers the daily amount of beta-carotene resulting to be missing in lactating women according to our study and necessary to reach levels associated with the beneficial observed effect on occurrence of mastitis, especially subclinical mastitis. In one embodiment, the dosage of beta-carotene as above described is adapted for a lactating woman.

The beta-carotene may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of beta-carotene as above described. In such embodiment, as it will be apparent to a person skilled in the art, the amount of beta-carotene contained in each serving of the composition according to the present invention will be divided by 1, 2 3 or 4 respectively.

In one embodiment, the composition according to the present invention is intended for consumption once or twice per day.

Vitamin C

Vitamin C may be incorporated in the composition of the invention as such or in the form of a physiologically acceptable salt and/or via any source comprising Vitamin C. For example ingredients may be selected in the group consisting of: ascorbic acid, sodium ascorbate and mixtures thereof.

As it is evident to the person skilled in the art, different ingredients may provide different amounts of Vitamin C in the composition according to the present invention, depending on the nature and amount of the ingredient used. It will be nonetheless routine work to the skilled person to calculate the amount of ingredient needed to provide the claimed amount of Vitamin C, based on the specification of the specific ingredient provided by the supplier.

In one embodiment, the dosage of Vitamin C is at least 100 mg/day. In a further embodiment, the dosage of Vitamin C is at least 105 mg/day. In a still further embodiment, the dosage of Vitamin C is ranging from 100 to 2000 mg/day, for example from 105 to 200 mg/day.

In one embodiment, the composition according to the present invention comprises at least 100 mg of Vitamin C. In a further embodiment, the composition according to the present invention comprises at least 105 mg of Vitamin C. In a still further embodiment, the composition comprises Vitamin C in an amount ranging from 100 to 2000 mg, for example 105 to 200 mg.

In such embodiment, the composition of the present invention delivers the daily amount of Vitamin C considered responsible for the beneficial observed effect on occurrence of mastitis, especially subclinical mastitis.

In one embodiment, the composition according to the present invention comprises at least 29 mg of Vitamin C. In a further embodiment, the composition according to the present invention comprises at least 35 mg of Vitamin C. In a still further embodiment, the composition comprises Vitamin C in an amount ranging from 29 to 2000 mg, for example 35 to 200 mg.

In such embodiment, the composition of the present invention delivers the daily amount of Vitamin C resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 14 mg of Vitamin C. In a further embodiment, the composition according to the present invention comprises at least 18 mg of Vitamin C. In a still further embodiment, the composition comprises Vitamin C in an amount ranging from 14 to 2000 mg, for example 18 to 200 mg.

In such embodiment, the composition of the present invention delivers the daily amount of Vitamin C resulting to be missing in lactating women according to our study and necessary to reach levels associated with the beneficial observed effect on occurrence of mastitis, especially subclinical mastitis.

In one embodiment, the dosage of vitamin C as above described is adapted for a lactating woman.

The vitamin C may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin C as above described. In such embodiment, as it will be apparent to a person skilled in the art, the amount of Vitamin C contained in each serving of the composition according to the present invention will be divided by 1, 2 3 or 4 respectively.

In one embodiment, the composition according to the present invention is intended for consumption once or twice per day.

Folate

Folate may be incorporated in the nutritional compositions of the invention as folic acid or in the form of a physiologically acceptable salt thereof (folate) or mixtures thereof.

In one embodiment, folate is of synthetic origin.

As it is evident to the person skilled in the art, different ingredients may provide different amounts of folate in the context of the present invention, depending on the nature and amount of the ingredient used. It will be nonetheless routine work to the skilled person to calculate the amount of ingredient needed to provide the claimed amount of folate, based on the specification of the specific ingredient provided by the supplier.

In one embodiment, the dosage of folate is at least 305 μg/day. In a further embodiment, the dosage of folate is at least 310 μg/day. In a still further embodiment, the dosage of folate is ranging from 305 to 1000 μg/day, for example from 310 to 600 μg/day.

In one embodiment, the composition according to the present invention comprises at least 305 μg of folate. In a further embodiment, the composition according to the present invention comprises at least 310 μg folate. In a still further embodiment, the composition comprises folate in an amount ranging 305 to 1000 μg, for example from 310 to 600 μg.

In such embodiment, the composition of the present invention delivers the daily amount of folate considered responsible for the beneficial observed effect on occurrence of mastitis, especially subclinical mastitis.

In one embodiment, the composition according to the present invention comprises at least 33 μg of folate. In a further embodiment, the composition according to the present invention comprises at least 40 μg folate. In a still further embodiment, the composition comprises folate in an amount ranging 33 to 1000 μg, for example from 40 to 600 μg.

In such embodiment, the composition of the present invention delivers the daily amount of folate resulting to be missing in lactating women according to our study and necessary to reach levels associated with the beneficial observed effect on occurrence of mastitis, especially subclinical mastitis.

In one embodiment, the composition according to the present invention comprises at least 66 μg of folate. In a further embodiment, the composition according to the present invention comprises at least 70 μg folate. In a still further embodiment, the composition comprises folate in an amount ranging 66 to 1000 μg, for example from 70 to 600 μg.

In such embodiment, the composition of the present invention delivers the daily amount of folate resulting to be missing in lactating women with subclinical mastitis according to our study and necessary to reach levels associated with the beneficial observed effect on occurrence of mastitis, especially subclinical mastitis.

In one embodiment, the dosage of folate as above described is adapted for a lactating woman.

The folate may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of folate as above described. In such embodiment, as it will be apparent to a person skilled in the art, the amount of folate contained in each serving of the composition according to the present invention will be divided by 1, 2 3 or 4 respectively.

In one embodiment, the composition according to the present invention is intended for consumption once or twice per day.

Potassium

Potassium may be provided in the context of the present invention as such or in the form of a physiologically acceptable salt and/or via any source comprising Potassium. For example ingredients may be selected in the group consisting of: potassium phosphate, potassium sulfate, potassium citrate, Potassium chloride, potassium aspartate, potassium bicarbonate, potassium gluconate and mixtures thereof.

In one embodiment, potassium is provided by potassium chloride.

As it is evident to the person skilled in the art, different ingredients may provide different amounts of potassium in the context of the present invention, depending on the nature and amount of the ingredient used. It will be nonetheless routine work to the skilled person to calculate the amount of ingredient needed to provide the claimed amount of potassium, based on the specification of the specific ingredient provided by the supplier.

In one embodiment, the dosage of potassium is at least 2800 mg/day. In a further embodiment, the dosage of potassium is at least 3000 mg/day. In a still further embodiment, the dosage of potassium is ranging from 2800 to 5000 mg/day, for example from 3000 to 4000 mg/day.

In one embodiment, the composition according to the present invention comprises at least 2800 mg of Potassium. In a further embodiment, the composition according to the present invention comprises at least 3000 mg of Potassium. In a still further embodiment, the composition comprises Potassium in an amount ranging from 2800 to 5000 mg, for example 3000 to 4000 mg.

In such embodiment, the composition of the present invention delivers the daily amount of Potassium considered responsible for the beneficial observed effect on occurrence of mastitis, especially subclinical mastitis.

In one embodiment, the composition according to the present invention comprises at least 480 mg of Potassium. In a further embodiment, the composition according to the present invention comprises at least 500 mg of Potassium. In a still further embodiment, the composition comprises Potassium in an amount ranging from 480 to 5000 mg, for example 500 to 4000 mg.

In such embodiment, the composition of the present invention delivers the daily amount of Potassium resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 240 mg of Potassium. In a further embodiment, the composition according to the present invention comprises at least 280 mg of Potassium. In a still further embodiment, the composition comprises Potassium in an amount ranging from 240 to 5000 mg, for example 280 to 4000 mg.

In such embodiment, the composition of the present invention delivers the daily amount of Potassium resulting to be missing in lactating women according to our study and necessary to reach levels associated with the beneficial observed effect on occurrence of mastitis, especially subclinical mastitis.

In one embodiment, the dosage of potassium as above described is adapted for a lactating woman.

The potassium may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of

Potassium as above described. In such embodiment, as it will be apparent to a person skilled in the art, the amount of Potassium contained in each serving of the composition according to the present invention will be divided by 1, 2 3 or 4 respectively.

In one embodiment, the composition according to the present invention is intended for consumption once or twice per day.

Vitamin B12 (Cobalamin)

Vitamin B12 may be incorporated in the composition of the invention as such or in the form of a physiologically acceptable salt and/or via any source comprising Vitamin B12. For example ingredients may be selected in the group consisting of: Cyanocobalamin, methylcobalamin, adenosylcobalamin and hydroxocobalamin.

As it is evident to the person skilled in the art, different ingredients may provide different amounts of Vitamin B12 in the composition according to the present invention, depending on the nature and amount of the ingredient used. It will be nonetheless routine work to the skilled person to calculate the amount of ingredient needed to provide the claimed amount of Vitamin B12, based on the specification of the specific ingredient provided by the supplier.

In one embodiment, the dosage of Vitamin B12 is at least 5 μg/day. In a further embodiment, the dosage of Vitamin B12 is at least 5.5 μg/day. In a still further embodiment, the dosage of Vitamin B12 is ranging from 5 to 250 μg/day, for example from 5.5 to 100 μg/day.

In one embodiment, the composition according to the present invention comprises at least 5 μg of Vitamin B12. In a further embodiment, the composition according to the present invention comprises at least 5.5 μg of Vitamin B12. In a still further embodiment, the composition comprises Vitamin B12 in an amount ranging from 5 to 250 μg, for example from 5.5 to 100 μg.

In such embodiment, the composition of the present invention delivers the daily amount of Vitamin B12 considered responsible for the beneficial observed effect on occurrence of mastitis, especially subclinical mastitis.

In one embodiment, the composition according to the present invention comprises at least 0.5 μg of Vitamin B12. In a further embodiment, the composition according to the present invention comprises at least 0.6 μg of Vitamin B12. In a still further embodiment, the composition comprises Vitamin B12 in an amount ranging from 0.5 to 250 μg, for example from 0.6 to 100 μg.

In such embodiment, the composition of the present invention delivers the daily amount of Vitamin B12 resulting to be missing in lactating women according to our study and necessary to reach levels associated with the beneficial observed effect on occurrence of mastitis, especially subclinical mastitis.

In one embodiment, the composition according to the present invention comprises at least 1.3 μg of Vitamin B12. In a further embodiment, the composition according to the present invention comprises at least 1.5 μg of Vitamin B12. In a still further embodiment, the composition comprises folate in an amount 1.3 to 250 μg, for example from 1.5 to 100 μg. In such embodiment, the composition of the present invention delivers the daily amount of Vitamin B12 resulting to be missing in lactating women with subclinical mastitis according to our study and necessary to reach levels associated with the beneficial observed effect on occurrence of mastitis, especially subclinical mastitis.

In one embodiment, the dosage of vitamin B12 as above described is adapted for a lactating woman.

The vitamin B12 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B12 as above described. In such embodiment, as it will be apparent to a person skilled in the art, the amount of Vitamin B12 contained in each serving of the composition according to the present invention will be divided by 1, 2 3 or 4 respectively.

In one embodiment, the composition according to the present invention is intended for consumption once or twice per day.

Vitamin B6

Vitamin B16 may be incorporated in the composition of the invention as such or in the form of a physiologically acceptable salt and/or via any source comprising Vitamin B6. For example ingredients may be selected in the group consisting of: pyridoxine (in the form of pyridoxine hydrochloride [HCl]) and pyridoxal 5′ phosphate (PLP).

As it is evident to the person skilled in the art, different ingredients may provide different amounts of Vitamin B6 in the composition according to the present invention, depending on the nature and amount of the ingredient used. It will be nonetheless routine work to the skilled person to calculate the amount of ingredient needed to provide the claimed amount of Vitamin B6, based on the specification of the specific ingredient provided by the supplier.

In one embodiment, the dosage of Vitamin B6 is at least 1.9 □g day. In a further embodiment, the dosage of Vitamin B6 is at least 2.0 mg/day. In a still further embodiment, the dosage of Vitamin B6 is ranging from 1.9 to 100 mg/day, for example from 2.0 to 50 mg/day.

In one embodiment, the composition according to the present invention comprises at least 1.9 mg of Vitamin B6. In a further embodiment, the composition according to the present invention comprises at least 2.0 mg of Vitamin B6. In a still further embodiment, the composition comprises Vitamin B6 in an amount ranging from 1.9 to 100 mg, for example from 2.0 to 50 mg.

In such embodiment, the composition of the present invention delivers the daily amount of Vitamin B6 considered responsible for the beneficial observed effect on occurrence of mastitis, especially subclinical mastitis.

In one embodiment, the composition according to the present invention comprises at least 0.35 mg of Vitamin B6. In a further embodiment, the composition according to the present invention comprises at least 0.4 mg of Vitamin B6. In a still further embodiment, the composition comprises Vitamin B6 in an amount ranging from 0.35 to 100 mg, for example from 0.4 to 50 mg.

In such embodiment, the composition of the present invention delivers the daily amount of Vitamin B6 resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 0.18 mg of Vitamin B6. In a further embodiment, the composition according to the present invention comprises at least 0.2 mg of Vitamin B6. In a still further embodiment, the composition comprises Vitamin B6 in an amount ranging from 0.18 to 100 mg, for example from 0.2 to 50 mg.

In such embodiment, the composition of the present invention delivers the daily amount of Vitamin B6 resulting to be missing in lactating women according to our study and necessary to reach levels associated with the beneficial observed effect on occurrence of mastitis, especially subclinical mastitis.

In one embodiment, the dosage of vitamin B6 as above described is adapted for a lactating woman.

The vitamin B6 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B6 as above described. In such embodiment, as it will be apparent to a person skilled in the art, the amount of Vitamin B6 contained in each serving of the composition according to the present invention will be divided by 1, 2 3 or 4 respectively.

Vitamin B5 (Pantothenic Acid)

Vitamin B5 may be incorporated in the composition of the invention as such or in the form of a physiologically acceptable salt and/or via any source comprising Vitamin B5. For example ingredients may be selected in the group consisting of: Pantothenic acid, pantethine, pantetheine and calcium pantothenate.

As it is evident to the person skilled in the art, different ingredients may provide different amounts of Vitamin B5 in the composition according to the present invention, depending on the nature and amount of the ingredient used. It will be nonetheless routine work to the skilled person to calculate the amount of ingredient needed to provide the claimed amount of Vitamin B5, based on the specification of the specific ingredient provided by the supplier.

In one embodiment, the dosage of Vitamin B5 is at least 5.3 mg/day. In a further embodiment, the dosage of Vitamin B5 is at least 5.5 mg/day. In a still further embodiment, the dosage of Vitamin C is ranging from 5.3 to 1500 mg/day, for example from 5.5 to 200 mg/day.

In one embodiment, the composition according to the present invention comprises at least 5.3 mg of Vitamin B5. In a further embodiment, the composition according to the present invention comprises at least 5.5 mg of Vitamin B5. In a still further embodiment, the composition comprises Vitamin B5 in an amount ranging from 5.3 to 1500 mg, for example from 5.5 to 200 mg.

In such embodiment, the composition of the present invention delivers the daily amount of Vitamin B5 considered responsible for the beneficial observed effect on occurrence of mastitis, especially subclinical mastitis.

In one embodiment, the composition according to the present invention comprises at least 0.85 mg of Vitamin B5. In a further embodiment, the composition according to the present invention comprises at least 0.9 mg of Vitamin B5. In a still further embodiment, the composition comprises Vitamin B5 in an amount ranging from 0.85 to 1500 mg, for example from 0.9 to 200 mg. In such embodiment, the composition of the present invention delivers the daily amount of Vitamin B5 resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 0.43 mg of Vitamin B5. In a further embodiment, the composition according to the present invention comprises at least 0.45 mg of Vitamin B5. In a still further embodiment, the composition comprises Vitamin B5 in an amount ranging from 0.43 to 1500 mg, for example from 0.45 to 100 mg.

In such embodiment, the composition of the present invention delivers the daily amount of Vitamin B5 resulting to be missing in lactating women according to our study and necessary to reach levels associated with the beneficial observed effect on occurrence of mastitis, especially subclinical mastitis.

In one embodiment, the dosage of vitamin B5 as above described is adapted for a lactating woman.

The vitamin B5 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B5 as above described. In such embodiment, as it will be apparent to a person skilled in the art, the amount of Vitamin B5 contained in each serving of the composition according to the present invention will be divided by 1, 2 3 or 4 respectively.

Vitamin B2 (Riboflavin)

Vitamin B2 may be incorporated in the composition of the invention as such or in the form of a physiologically acceptable salt and/or via any source comprising Vitamin B2. For example ingredients may be selected in the group consisting of: riboflavin and riboflavin 5′-monophosphate.

As it is evident to the person skilled in the art, different ingredients may provide different amounts of Vitamin B2 in the composition according to the present invention, depending on the nature and amount of the ingredient used. It will be nonetheless routine work to the skilled person to calculate the amount of ingredient needed to provide the claimed amount of Vitamin B2, based on the specification of the specific ingredient provided by the supplier.

In one embodiment, the dosage of Vitamin B2 is at least 1.8 mg/day. In a further embodiment, the dosage of Vitamin B2 is at least 2.0 mg/day. In a still further embodiment, the dosage of Vitamin B2 is ranging from 1.8 to 5 mg/day, for example from 2.0 to 4 mg/day.

In one embodiment, the composition according to the present invention comprises at least 1.8 mg of Vitamin B2. In a still further embodiment, the composition comprises Vitamin B2 in an amount ranging from 1.8 to 5 mg, for example from 1.8 to 2 mg.

In such embodiment, the composition of the present invention delivers the daily amount of Vitamin B2 considered responsible for the beneficial observed effect on occurrence of mastitis, especially subclinical mastitis.

In one embodiment, the composition according to the present invention comprises at least 0.3 mg of Vitamin B2. In a still further embodiment, the composition comprises Vitamin B2 in an amount ranging from 0.3 to 5 mg, for example from 0.35 to 2 mg.

In such embodiment, the composition of the present invention delivers the daily amount of Vitamin B2 resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 0.15 mg of Vitamin B2. In a still further embodiment, the composition comprises Vitamin B2 in an amount ranging from 0.15 to 5 mg, for example from 0.18 to 2 mg. In such embodiment, the composition of the present invention delivers the daily amount of Vitamin B2 resulting to be missing in lactating women according to our study and necessary to reach levels associated with the beneficial observed effect on occurrence of mastitis, especially subclinical mastitis.

In one embodiment, the dosage of vitamin B2 as above described is adapted for a lactating woman.

The vitamin B2 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B2 as above described. In such embodiment, as it will be apparent to a person skilled in the art, the amount of Vitamin B2 contained in each serving of the composition according to the present invention will be divided by 1, 2 3 or 4 respectively.

Vitamin B1 (Thiamin)

Vitamin B1 may be incorporated in the composition of the invention as such or in the form of a physiologically acceptable salt and/or via any source comprising Vitamin B1. For example ingredients may be selected in the group consisting of: thiamin mononitrate and thiamin hydrochloride.

As it is evident to the person skilled in the art, different ingredients may provide different amounts of Vitamin B1 in the composition according to the present invention, depending on the nature and amount of the ingredient used. It will be nonetheless routine work to the skilled person to calculate the amount of ingredient needed to provide the claimed amount of Vitamin B1, based on the specification of the specific ingredient provided by the supplier.

In one embodiment, the dosage of Vitamin B1 is at least 1.6 mg/day. In a further embodiment, the dosage of Vitamin B1 is at least 1.8 mg/day. In a still further embodiment, the dosage of Vitamin B1 is ranging from 1.6 to 500 mg/day, for example from 1.8 to 5 mg/day.

In one embodiment, the composition according to the present invention comprises at least 1.6 mg of Vitamin B1. In a still further embodiment, the composition comprises Vitamin B1 in an amount ranging from 1.6 to 500 mg, for example from 1.8 to 5 mg.

In such embodiment, the composition of the present invention delivers the daily amount of Vitamin B1 considered responsible for the beneficial observed effect on occurrence of mastitis, especially subclinical mastitis.

In one embodiment, the composition according to the present invention comprises at least 0.29 mg of Vitamin B1. In a still further embodiment, the composition comprises Vitamin B1 in an amount ranging from 0.29 to 500 mg, for example from 0.3 to 5 mg. In such embodiment, the composition of the present invention delivers the daily amount of Vitamin B1 resulting to be missing in lactating women with subclinical mastitis according to our study and necessary to reach levels associated with the beneficial observed effect on occurrence of mastitis, especially subclinical mastitis.

In one embodiment, the composition according to the present invention comprises at least 0.14 mg of Vitamin B1. In a still further embodiment, the composition comprises Vitamin B1 in an amount ranging from 0.14 to 500 mg, for example from 0.15 to 5 mg.

In such embodiment, the composition of the present invention delivers the daily amount of Vitamin B1 resulting to be missing in lactating women according to our study and necessary to reach levels associated with the beneficial observed effect on occurrence of mastitis, especially subclinical mastitis.

In one embodiment, the dosage of vitamin B1 as above described is adapted for a lactating woman.

The vitamin B1 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B1 as above described. In such embodiment, as it will be apparent to a person skilled in the art, the amount of Vitamin B1 contained in each serving of the composition according to the present invention will be divided by 1, 2 3 or 4 respectively.

Mastitis

Mastitis is an inflammation of the mammary gland tissue, which can be classified as sub-clinical or clinical depending on the degree of inflammation.

Clinical mastitis is a form of mastitis associated with reduced milk secretion, visible signs of inflammation of the breast and, changes in the appearance of milk, which may be accompanied by systemic signs. Sub-clinical mastitis is a form of mastitis characterised by reduced milk secretion and a high milk bacterial count in the absence of evident inflammatory changes, including pain (Fernandez, L. et al. (2014) Beneficial Microbes 5: 169-183).

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain.

Staphylococcus infections, in particular S. aureus and S. epidermidis infections, are understood to be a primary cause of mastitis.

Mastitis can result in curtailment or even lack of initiation of breast-feeding of an infant. Furthermore, the composition of breast milk may change during mastitis, for example increasing in content of sodium and inflammatory mediators, which may adversely affect the nutrition provided to the infant.

In one embodiment of the present invention, mastitis is sub-clinical mastitis.

Combinations with Other Nutrients

In one embodiment of the present invention, a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof, may be used in combination with other nutrients to treat prevent and/or reduce the risk of mastitis, for example clinical and/or subclinical mastitis.

In one embodiment, a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof, may be used in combination with
(a) a mineral selected in the group consisting of: iron, manganese, magnesium, copper, calcium, phosphorus, zinc and selenium;
(b) an n-3 fatty acid, preferably wherein the nutrient is in combination with a fatty acid selected from the group consisting of docosahexaenoic acid (DHA) and 18:3 n-3 octadecatrienoic acid (alpha-linolenic acid);
(c) a protein selected from the group consisting of alpha-lactalbumin, lactoferrin and albumin;
(d) Vitamin E; and/or
(e) phosphatidylcholine and/or lecithin.

In one embodiment, a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof, may be used in combination with a mineral selected in the group consisting of: iron, manganese, magnesium, copper, calcium, phosphorus and a combination of two or more thereof.

In one embodiment, a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof, may be used in combination with
(a) a mineral selected in the group consisting of: iron, manganese, magnesium, copper, calcium, phosphorus, zinc and selenium;
(b) an n-3 fatty acid, preferably wherein the nutrient is in combination with a fatty acid selected from the group consisting of docosahexaenoic acid (DHA) and 18:3 n-3 octadecatrienoic acid (alpha-linolenic acid);
(c) a protein selected from the group consisting of alpha-lactalbumin, lactoferrin and albumin;
(d) Vitamin E; and/or
(e) phosphatidylcholine and/or lecithin;

to treat prevent and/or reduce the risk of mastitis, for example clinical and/or subclinical mastitis.

Minerals

In one embodiment, a nutrient selected from the group consisting of: beta-carotene, fiber, Vitamin C, Folate, Potassium, Vitamin B1, Vitamin B2, Vitamin B5, Vitamin B6, Vitamin B12 and a combination of two or more thereof may be combined with a mineral selected from the group consisting of iron, manganese, magnesium, and a combination of two of more thereof, for use in treating or preventing mastitis in a subject.

In one embodiment, the nutrient is in combination with one or more further minerals selected from the group consisting of magnesium, manganese, iron, copper, zinc, selenium, calcium and phosphorus.

The minerals may be used in any form suitable for ingestion by animals, preferably humans (e.g. are non-toxic). The minerals may be used, for example in compositions such as nutritional compositions, in any appropriate amount. The skilled person will be able to determine appropriate amounts depending on the desired dosage of the mineral. Dosages may depend on factors such as the age, size and health status of the woman to whom they are administered, on her lifestyle, as well as on her genetic heritage. Dosages may be in line with the recommended daily intakes (RDA) developed by organisations such as the Food and Nutrition Board of the National Academy of Sciences.

The skilled person can readily determine an appropriate dose of one of the agents of the invention to administer to a subject without undue experimentation. Typically, a physician will determine the actual dosage that will be most suitable for an individual subject and it will depend on a variety of factors including the activity of the specific agent employed, the metabolic stability and length of action of that agent, the age, body weight, general health, sex, diet, mode and time of administration, rate of excretion, drug combination, the severity of the particular condition and the individual undergoing therapy. There can of course be individual instances where higher or lower dosage ranges are merited.

In one embodiment, the dosage of iron is about 2.7-45, 5-25 or 9-10 mg/day. A dosage of about 9-10 mg/day may be preferred for breast-feeding women.

In another embodiment, the dosage of iron is about 30-60 mg/day. A dosage of about 30-60 mg/day may be preferred for pregnant women.

In one embodiment, the dosage of iron is at least 9.1 mg/day. In a further embodiment, the dosage of iron is at least 9.5 mg/day. In a still further embodiment, the dosage of iron is ranging from 9.5 to 60 mg/day, for example from 9.5 to 50 mg/day, for example 9.5 to 40 mg/day.

In one embodiment, the dosage of iron for a lactating woman is at least 9.1 mg/day. In a further embodiment, the dosage of iron is at least 9.5 mg/day. In a still further embodiment, the dosage of iron is ranging from 9.5 to 60 mg/day, for example from 9.5 to 30 mg/day, for example 9.5 to 20 mg/day.

The iron may be comprised in any form suitable for ingestion by a woman such as a pregnant woman, a woman trying to conceive or a lactating woman. For example, iron may be comprised in the form of iron sulfate, iron citrate, iron choline citrate, iron ammonium citrate, iron chloride, iron fumarate, iron gluconate, iron pyroposphate or a mixture thereof.

In one embodiment, the dosage of manganese is about 1.8-11, 2-3 or 2.5-2.7 mg/day. A dosage of about 2.5-2.7 mg/day may be preferred for breast-feeding women. A dosage of about 1.9-2.1 mg/day may be preferred for pregnant women.

In one embodiment, the dosage of manganese is at least 2.1 mg/day. In a further embodiment, the dosage of manganese is at least 2.3 mg/day. In a still further embodiment, the dosage of manganese is ranging from 2.1 to 4 mg/day, for example from 2.3 to 3.5 mg/day.

In one embodiment, the dosage of manganese for a lactating woman is at least 2.1 mg/day. In a further embodiment, the dosage of manganese is at least 2.3 mg/day. In a still further embodiment, the dosage of manganese is ranging from 2.1 to 4 mg/day, for example from 2.3 to 3.5 mg/day.

The manganese may be comprised in any form suitable for ingestion by a woman such as a pregnant woman, a woman trying to conceive or a lactating woman. For example, manganese may be comprised in the form of manganese gluconate, manganese sulfate, manganese ascorbate, manganese amino acid chelates, manganese aspartate, manganese picolinate, manganese fumarate, manganese malate, manganese succinate, manganese citrate or a mixture thereof.

In one embodiment, the dosage of magnesium is about 35-350, 200-350 or 300-350 mg/day. A dosage of about 300-350 mg/day may be preferred for breast-feeding women.

In one embodiment, the dosage of magnesium is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

In one embodiment, the dosage of magnesium for a lactating woman is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

The magnesium may be comprised in any form suitable for ingestion by a woman such as a pregnant woman, a woman trying to conceive or a lactating woman. For example, magnesium may be comprised in the form of magnesium chloride, magnesium citrate, magnesium sulfate, magnesium oxide, magnesium hydroxide, magnesium amino acid chelates (e.g. chelates of glycinate, lysinate, orotate, taurate, aspartate, threonate and/or malate) or a mixture thereof.

In one embodiment, the dosage of copper is about 0.1-10, 0.1-2 or 0.5-1.5 mg/day.

In one embodiment, the dosage of copper is at least 1.250 mg/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

In one embodiment, the dosage of copper for a lactating woman is at least 1.250 mg/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

The copper may be comprised in any form suitable for ingestion by a woman such as a pregnant woman, a woman trying to conceive or a lactating woman. For example, copper may be comprised in the form of copper oxide, copper chloride, copper gluconate, copper sulfate, copper amino acid chelates or a mixture thereof.

In one embodiment, the dosage of zinc may be about 5-40, 7-13 or 9.5-12 mg/day.

The zinc may be comprised in any form suitable for ingestion by a woman such as a pregnant woman, a woman trying to conceive or a lactating woman. For example, zinc may be comprised in the form of zinc acetate, zinc chloride, zinc citrate, zinc gluconate, zinc lactate, zinc oxide, zinc sulfate, zinc carbonate or a mixture thereof.

In one embodiment, the dosage of selenium may be about 20-400, 25-250, 26-85 or 60-70 μg/day.

The selenium may be comprised in any form suitable for ingestion by a woman such as a pregnant woman, a woman trying to conceive or a lactating woman. For example, selenium may be comprised in the form of sodium selenite, sodium hydrogen selenite or a mixture thereof.

In one embodiment, the dosage of calcium is about 100-2500, 500-2000 or 1000-1500 mg/day.

In one embodiment, the dosage of calcium is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day.

In one embodiment, the dosage of calcium for a lactating woman is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day.

The calcium may be comprised in any form suitable for ingestion by a woman such as a pregnant woman, a woman trying to conceive or a lactating woman. For example, calcium may be comprised in the form of calcium citrate, calcium carbonate or a mixture thereof.

In one embodiment, the dosage of phosphorous is about 70-4000, 100-1500 or 250-1250 mg/day.

In one embodiment, the dosage of phosphorus is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

In one embodiment, the dosage of phosphorus for a lactating woman is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

The phosphorous may be comprised in any form suitable for ingestion by a woman such as a pregnant woman, a woman trying to conceive or a lactating woman. For example, phosphorous may be comprised in the form of sodium phosphate.

In a further embodiment of the present invention, the dosage of iron is ranging from 9.5 to 60 mg/day, for example from 9.5 to 45 mg/day, for example from 9.5 to 30 mg/day, for example 9.5 to 20 mg/day; the dosage of manganese is ranging from 2.1 to 4 mg/day, for example from 2.3 to 3.5 mg/day; the dosage of magnesium is ranging from 270 to 350 mg/day, for example from 300 to 350 mg/day; the dosage of copper is ranging from 1.250 to 10 mg/day, for example from 1.30 to 2 mg/day, for example from 1.30 to 1.50 mg/day; the dosage of calcium is ranging from 750 to 2500 mg/day, for example from 850 to 2000 mg/day, for example from 900 to 1500 mg/day; and the dosage of phosphorus is ranging from 1300 to 4000 mg/day, for example from 1300 to 2000 mg/day, for example from 1300 to 1500 mg/day. In such embodiment, the subject receiving the mineral combination or composition comprising it is for example a lactating woman.

Vitamins, Fatty Acids and Proteins

In one embodiment, The nutrient selected from the group consisting of: beta-carotene, fiber, Vitamin C, Folate, Potassium and a combination of two or more thereof may be used in combination with further agents, in particular vitamin E, n-3 fatty acids (preferably selected from the group consisting of docosahexaenoic acid (DHA) and 18:3 n-3 octadecatrienoic acid (alpha-linolenic acid)) and/or a protein selected from the group consisting of alpha-lactalbumin, lactoferrin and albumin for treating or preventing mastitis in a subject.

Such agents (vitamin E, n-3 fatty acids, alpha-lactalbumin, lactoferrin and albumin) may be used in any form suitable for ingestion by animals, preferably humans (e.g. are non-toxic). The agents may be used, for example in compositions such as nutritional compositions, in any appropriate amount. The skilled person will be able to determine appropriate amounts depending on the desired dosage of the agent. Dosages may depend on factors such as the age, size and health status of the woman to whom they are administered, on her lifestyle, as well as on her genetic heritage. Dosages may be in line with the recommended daily intakes (RDA) developed by organisations such as the Food and Nutrition Board of the National Academy of Sciences.

In one embodiment, the dosage of vitamin E is about 11-1000, 7.5-300 or 11-19 mg/day.

In one embodiment, the dosage of vitamin E is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day, for example from 9.5 to 19 mg/day.

In one embodiment, the dosage of vitamin E for a lactating woman is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day, for example from 9.5 to 19 mg/day.

The vitamin E may be, for example, in the form of a tocopherol or a mixture of different tocopherols. For example, the vitamin E may be alpha-tocopherol, gamma-tocopherol or a mixture of alpha-tocopherol and gamma-tocopherol.

The vitamin E may be comprised in any form suitable for ingestion by a woman such as a pregnant woman, a woman trying to conceive or a lactating woman, for example, alpha-tocopherol and/or gamma-tocopherol, and/or may be comprised in the form of tocopherol concentrate mix, L-vitamin E, D,L-vitamin E, tocopherols mixed pure, D,L-alpha-tocopherol, D,L-alpha tocopheryl acetate, tocopherol rich extract or a mixture thereof.

In one embodiment, the vitamin E is alpha-tocopherol.

In one embodiment, the dosage of docosahexaenoic acid (DHA) is less than or equal to 1000 mg/day, preferably about 500-1000 mg/day.

In one embodiment, the dosage of alpha-linolenic acid is less than or equal to 2000 mg/day, preferably about 500-1000 mg/day.

In one embodiment, the dosage of phosphatidylcholine is about 1500-1750 mg/day. In one embodiment, the dosage of lecithin is about 1500-1750 mg/day.

In one embodiment, the dosage of lactoferrin is about 5-500 mg/day, preferably about 100-500 mg/day.

With respect to dosages defined herein as amounts per daily dose the amount of nutrient in a composition administered to the subject may vary depending upon whether it is intended to be consumed once a day, or more or less frequently.

Methods of Treatment

The term “combination”, or terms “in combination”, “used in combination with” or “combined preparation” as used herein may refer to the combined administration of two or more agents simultaneously, sequentially or separately.

The “mix” as used in the present document refers to the concomitant presence of relevant ingredients or molecules. The mix is not limited to the actual mixing of the relevant ingredients or molecules. For example, a mineral and the probiotic used in the invention can be present in a nutritional composition of the invention (i.e. the “mix” is present) without having been actually mixed together separately at any stage of the manufacturing of the composition.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

It is to be appreciated that all references herein to treatment include curative, palliative and prophylactic treatment. The treatment of mammals, particularly humans, is preferred. Both human and veterinary treatments are within the scope of the invention.

The minerals, fatty acids, proteins, combinations and compositions disclosed herein may be administered to a woman desiring to get pregnant, to a pregnant woman and/or to a lactating woman.

If administration is to a woman desiring to get pregnant, administration may be for example during at least 1, 2, 3 or 4 months preceding the pregnancy or desired pregnancy.

If administration is to a pregnant woman, administration may be for example for at least 4, at least 8, at least 12, at least 16, at least 20, at least 24, at least 28 or at least 36 weeks during pregnancy. As the nutritional requirements increase in the second and third trimester of pregnancy, it may be particularly beneficial if administration is throughout the second and/or third trimester of pregnancy.

Administration pre-pregnancy and/or during pregnancy may enable a woman to build up a store of one or more of the nutrients before lactation.

If administration is to a lactating woman, administration may be for example for any part of the lactation period for example up to 2 years, up to 1 year, up to 9, 8, 7, 6, 5, 4, 3, 2 or 1 months post birth.

In one embodiment, administration is to a woman desiring to get pregnant, to a pregnant woman and/or to a lactating woman.

Composition

The term “maternal nutritional composition” as used herein refers to any composition that has been specifically manufactured for consumption by a pregnant woman, a woman trying to conceive or a lactating woman, or a composition that is specifically marketed at pregnant women, women trying to conceive or lactating (e.g. breast-feeding) women.

The maternal nutritional composition may be, for example, a food product, a functional food product, a drink (beverage), a dairy product or dairy substitute product, a pharmaceutical formulation or a supplement.

The term “dairy product” as used herein refers to food products produced from animals such as cows, goats, sheep, yaks, horses, camels and other mammals. Examples of dairy products are low-fat milk (e.g. 0.1%, 0.5% or 1.5% fat milk), fat-free milk, milk powder, whole milk, whole milk products, butter, buttermilk, buttermilk products, skim milk, lactose-free products, high milk-fat products, condensed milk, crème fraiche, cheese, ice cream and confectionery products, probiotic drinks or probiotic yoghurt-type drinks. A dairy substitute product may be a soya, almond or vegetable-based dairy substitute, e.g. a milk or yoghurt substitute.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “beverage product” as used herein refers to a nutritional product in liquid or semi-liquid form that may be safely consumed by an individual. The beverage product may be a water-based product, such as a product in which the agents of the invention are dissolved or suspended in water.

The term “food product” as used herein refers to any kind of product that may be safely consumed by a woman, in particular a pregnant woman, a woman trying to conceive or a lactating (e.g. breast-feeding) woman. Said food product may be in solid, semi-solid or liquid form and may comprise one or more nutrients, foods or nutritional supplements. For example, the food product may further comprise one or more of the following nutrients and micronutrients: a source of protein, a source of lipid, a source of carbohydrate, vitamins and minerals. The composition may also contain anti-oxidants, stabilisers (when provided in solid form) or emulsifiers (when provided in liquid form).

The term “functional food product” as used herein refers to a food product providing an additional health-promoting or disease-preventing function to the individual. Food products and functional food products include, for example, cereal-based products, yoghurts or other milk-derived products and bars.

The term “supplement” as used herein refers to a nutritional product that provides nutrients (e.g. vitamins and/or minerals) to an individual that may otherwise not be consumed in sufficient quantities by said individual. Supplements may be, for example, provided in the form of a pill, a tablet, a lozenge, a chewy capsule or tablet, a capsule, or a powder supplement that can be, for example, dissolved in water or milk, or sprinkled on food. Supplements typically provide selected nutrients without providing a significant portion of the overall nutritional needs of a subject. Typically supplements do not represent more than 0.1%, 1%, 5%, 10% or 20% of the daily energy need of a subject. In the context of the present invention the subject may be, for example, a woman trying to get pregnant, a pregnant woman and/or a lactating woman.

The term “pregnancy supplement” as used herein refers to a supplement that is specifically formulated for administration to a woman who is trying to conceive and/or to a woman who is pregnant, or marketed towards a woman who is trying to conceive and/or a woman who is pregnant.

The term “lactation supplement” as used herein refers to a supplement that is specifically formulated for administration to a woman who is lactating, or marketed toward a woman who is lactating. Consumption of lactation supplements may be advised to commence during pregnancy.

The compositions of the invention may also comprise ingredients commonly used in maternal nutritional compositions. Non-limiting examples of such ingredients include: probiotics, lipids, carbohydrates, pharmaceutically-active agents and conventional additives, such as anti-oxidants, stabilisers, emulsifiers, acidulants, thickeners, buffers or agents for pH adjustment, chelating agents, colourants, excipients, flavour agents, osmotic agents, pharmaceutically-acceptable carriers, preservatives, sugars, sweeteners, texturisers, emulsifiers and water.

It may also be beneficial if the compositions of the invention comprise probiotics. Probiotics may help nutrients pass through the gut.

Probiotic

The term “probiotic”, “a probiotic”, “probiotics” as used interchangeably herein refers to live probiotic bacteria, non-replicating probiotic bacteria, dead probiotic bacteria, non-viable probiotic bacteria, fragments of probiotic bacteria, such as DNA, metabolites of probiotic bacteria, cytoplasmic compounds of probiotic bacteria, cell wall materials of probiotic bacteria, culture supernatants of probiotic bacteria, and combinations of any of the foregoing.

The probiotic may be live probiotic bacteria, non-replicating probiotic bacteria, dead probiotic bacteria, non-viable probiotic bacteria and any combination thereof.

The mix and/or composition of the invention comprises a particular Lactobacillus Fermentum strain, namely Lactobacillus Fermentum CECT5716. The strain Lactobacillus Fermentum CECT5716 is known and its genone has been sequenced (“Complete Genome Sequence of Lactobacillus Fermentum CECT 5716, a Probiotic Strain Isolated from Human Milk”—JOURNAL OF BACTERIOLOGY, September 2010, p. 4800, vol. 192, No 18, by Esther Jimenez, Susana Langa, Virginia Martin, Rebeca Arroyo, Rocio Martin, Leonides Fernandez, and Juan M. Rodriguez). The strain has been deposited under the reference CECT-5716 at the Coleccion Espanola de Cultivos Tipo, under the Budapest treaty (address: c/Catedrático Agustin Escardino, 9. 46980 Paterna (Valencia), Spain). The strain is the subject of the patent EP1565547B1 which comprises claims to the strain L. fermentum CECT-5716 itself (applicant: Biosearch S. A., Camino de Purchil 66, 18004 Granada/ES). L. fermentum CECT-5716 is commercialized by Biosearch S. A., in particular under the tradename/trademark Hereditum LC-40 ®. The strain L. fermentum CECT-5716 has also been fully described in a request for a US GRAS approval by the US Food & Drug Administration (approval for ingredients “Generally Recognized as Safe”) under reference number GRN-531, publically available under the following link:

https://www.accessdata.fda.gov/scripts/fdcc/?set=GRASNotices&id=531&sort=GRN No&order=DESC&startrow=1&type=basic&search=531.

The strain L. fermentum CECT-5716 has been well studied, and has in particular been linked to a reduction in the pathogenic load and to a positive effect on mastitis. See in particular the following scientific articles:
Study protocol: evaluation of the probiotic Lactobacillus Fermentum CECT5716 for the prevention of mastitis in breastfeeding women: a randomised controlled trial; Bond et al; BMC Pregnancy and Childbirth (2017) 17:148 DOI 10.1186/s12884-017-1330-8
Treatment of Infectious Mastitis during Lactation: Antibiotics versus Oral Administration of Lactobacilli Isolated from Breast Milk, by Rebeca Arroyo, Virginia Martin, Antonio Maldonado, Esther Jiménez, Leónides Fernández, Juan Miguel Rodriguez; Clinical Infectious Diseases, Volume 50, Issue 12, 15 Jun. 2010, Pages 1551-1558, https://doi.org/10.1086/652763.

The effect on mastitis has however been sometimes challenged (see in particular the scientific opinion by an EFSA panel dated Jun. 27, 2007—“Lactobacillus Fermentum CECT 5716 and a reduction of the Staphylococcus load in breast milk which reduces the risk of infectious mastitis: evaluation of a health claim pursuant to Article 14 of Regulation (EC) No 1924/2006”, EJ EFSA Journal, doi: 10.2903/j.efsa.2017.4917). The inventors may hypothetize that the evidences in some of the studies may have been partly or entirely shaded by the non controlled intake other nutrients or minerals. On contrary the present invention builds on the synergy between the specific strain in a context of a relatively significant amount of selected other nutrients or minerals, and especially in a population of women at risk of mastitis or sub-clinical mastitis (i.e. partly deficient in those nutrients/minerals). Without being bound by the theory it is believed that the effect of the CECT-5716 probiotic on balancing out potential pathogenic bacteria, (including influencing their growth), and the beneficial effect of selected nutrients/minerals, for example on the immune system and/or the inflammatory status, can synergize to reveal and/or enhance a better handling against mastitis—both at the prevention level avoiding the occurrence of sub-clinical mastitis and at the direct reduction of the occurrence or severity (treatment level).

In one embodiment the probiotic strain L. fermentum CECT-5716 may be present in the mix or in the composition of the invention in an amount of 10² cfu to 10¹² cfu per g of composition (or mix). Alternatively 10³ to 10¹⁶ cfu/g or 10⁵ to 10⁸ cfu/g or 10⁶ to 10⁷ cfu/g.

In one embodiment one or more other probiotics are also present in the mix or composition of the invention (“other probiotics” meaning “other than Fermentum CECT-5716”, which may still be present as per the present invention).

Generally the probiotics may help establishing a healthy gut microbiota and strengthen natural immune defenses. The probiotics also stimulate a development of the immune system at introduction of weaning food and prevent diarrhea. By stimulating the immune system the probiotics can synergize with the other essential components of the invention.

Examples of suitable probiotic micro-organisms include bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus.

Specific examples of suitable probiotic micro-organisms are: Saccharomyces cereviseae, Bacillus coagulans, Bacillus licheniformis, Bacillus subtilis, Bifidobacterium bifidum, Bifidobacterium infantis, Bifidobacterium adolescentis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

Probiotic bacterial strains useful in the context of the present invention may include Lactobacillus rhamnosus ATCC 53103 obtainable from Valio Oy of Finland under the trade mark LGG, Lactobacillus rhamnosus CGMCC 1.3724, Lactobacillus paracasei CNCM 1-2116, Bifidobacterium lactis CNCM 1-3446 sold inter alia by the Christian Hansen company of Denmark under the trade mark Bb 12 and Bifidobacterium longum ATCC BAA-999 sold by Morinaga Milk Industry Co. Ltd. of Japan under the trade mark BB536, Bifidobacterium adolescentis IVS-1 (Danwell Technology, Garden Grove, Calif.), Bifidobacterium adolescentis MG10502, (obtainable from Belgian Coordinated Collection of Microorganisms (BCCM/LMG), Bifidobacterium infantis Rosen-33, Bifidobacterium infantis (in particular strain LMG 8811, species name: Bifidobacterium longum ATCC 1569,

In one embodiment of the invention the strain mentioned in the above paragraph are used in replacement of the strain L. fermentum CECT-5716, in combination with the nutrient/mineral of the invention.

Subject

The term “subject” as used herein refers to either a human or non-human animal. The non-human animal may be, for example, a livestock animal or a companion animal.

A “companion animal” is any domesticated animal, and includes, without limitation, cats, dogs, rabbits, guinea pigs, ferrets, hamsters, mice, gerbils, horses, cows, goats, sheep, donkeys, pigs and the like.

In one embodiment, the subject is a human subject. In another embodiment, the subject is a companion animal. Preferably, the subject is a human.

In one embodiment, the subject is at risk of mastitis and/or subclinical mastitis. In another embodiment, the subject is a lactating animal.

In one embodiment, the human subject is a woman. In a further embodiment, the human subject is a lactating woman. In another embodiment, the human subject is a pregnant woman. It is believed that the compositions and mix of the invention can have a long term effect which pre-establishes adequate body conditions during pregnancy in order to prevent or treat mastitis during later lactation.

In one embodiment, the human subject is a woman having mastitis and/or of subclinical mastitis.

In a still further embodiment, the human subject is a woman at risk of mastitis and/or of subclinical mastitis.

In another embodiment, the human subject is a lactating woman at risk of mastitis and/or of subclinical mastitis.

In an additional embodiment, the subject is a European lactating woman. In an additional embodiment, the subject is a Caucasian lactating woman. In an additional embodiment, the subject is a European Caucasian lactating woman.

Treating and Preventing

The term “prevent” as used herein includes prevention and reducing the risk of a condition.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature.

### EXAMPLES

### Example 1

Methods

Study Population

This study used data from ‘ATLAS’, a longitudinal, observational study across seven European countries between December 2012 and January 2016. The study was approved by the institutional and local ethical boards for each center and was registered at ClincalTrials.gov with identifier NCT01894893. Maternal and infant demographics, anthropometry, and medical history were collected by trained and certified research nurses and assistants.

Human milk was collected from 305 women in 7 European countries. Of those, 185 provided information on dietary intake. 8 women were further excluded due to missing information, resulting in a final sample size of 177 women. Written informed consent was obtained from all women in their respective local languages.

SCM Analysis

Milk samples were obtained using an electric breast pump (Medela Symphony, Switzerland) from the same breast throughout the study period, at 11:00 h±2:00 h to avoid circadian influence. Samples were first frozen at −18° C. until delivery to the Nestle Research Centre (Lausanne, Switzerland) and then at −80° C. for further analysis.

SCM was assessed in early lactation, at visits 1 (0-3 days postpartum), 2 (17 days postpartum±3 days), and 3 (30 days postpartum±3 days). SCM was defined as having a sodium potassium ratio (Na/K) in breastmilk higher than 0.6 at any of the three visits.

Dietary Intake Data

Dietary intake was assessed with 3-day food diaries at visits 2 (V2) and 3 (V3). The dietary information was then translated to nutrient and food group intakes by Nutrilog using the French food group classification and nutrient composition database (CIQUAL).

Diets which contained less than 1074.8 kcal or more than 4776.9 kcal of energy were considered outliers and were removed. After removing the outlier diets from the dataset, we considered each visit in V2-V3 in turn and the subset of subjects who attended that visit. For each visit, we removed a subject and all associated dietary information from a given visit if fewer than two non-outlier diets were reported for that subject across the three-day survey period for that visit. For example, if a subject 51 attended visit V2 and reported one outlier diet and one non-outlier diet for that visit, then we removed S1 and all her reported diets for V2 from the dataset. 177 subjects who attended at least one visit were retained for analysis.

Once the mean daily consumption was calculated, it was then normalized by the mean daily energy intake for that visit in kcal/day. This adjusted consumption was then averaged over all visits that each individual subject attended within the subset. As a final step, we then performed normalization to zero mean and standard deviation of 1. An example pipeline for a subject who attended all visits in S2 (visits V2-V3)

Dietary reference values for nutrients intake for lactating women were extracted from European Food Safety Authority (EFSA)'s summary report on Dietary Reference Values for nutrients.

### Statistical Analysis

1) We used multivariable regression to examine the association between nutrient intake in relation to SCM. Wilcox-test was used to calculate the p-values.

Analyses were run with R.

Results are reported in Table 1.

2) We also used multivariable regression to examine the association between nutrient intake in relation to SCM. The statistical model has SCM status, Country and Mode of Delivery as covariates and contrast estimates were calculated to show the differences between SCM group and the No SCM group. A logarithmic transformation was applied as the nutrient intake data generally has a skewed distribution.

Analyses were run with the statistical software R version 3.2.1 and packages mass and contrast were used for modelling and estimation. Results are reported in Table 2.

Results

Table 1 reports the median intake of certain nutrients for women with SCM (i.e. those with human milk sodium potassium (Na/K) ratio >0.6 during any of the following visits: days 2, 17, and 30), and for women with no SCM (i.e. those with no SCM are defined as having a Na/K ratio ≤0.6 during any of the following visits: days 2, 17, and 30).

These data show that in the group of women with subclinical mastitis, certain nutrients (namely beta-carotene, fiber, Vitamin C, Folate and Potassium) were present at lower amounts in the diet as compared to the diet of women not having subclinical mastitis.

Similarly, data show that in the group of women with subclinical mastitis vitamin E and iron, manganese, magnesium, copper, calcium, phosphorous were present at lower amounts in the diet as compared to the diet of women not having subclinical mastitis.

These findings thus provide evidence that supplementation of one or more of such nutrients in the diet can prevent and/or treat subclinical mastitis.

Table 2 reports the geometric mean for certain nutrients in women with SCM (i.e. those with human milk sodium potassium (Na/K) ratio >0.6 during any of the following visits: days 2, 17, and 30), and in women with no SCM (i.e. those with no SCM are defined as having a Na/K ratio ≤0.6 during any of the following visits: days 2, 17, and 30).

These data confirm the findings reported in table 1 for nutrients abovementioned and additionally show that in the group of women with subclinical mastitis certain additional nutrients (namely thiamin, riboflavin, Pantothenic acid, Vitamin B6 and Vitamin B12) were present at lower amounts in the diet as compared to the diet of women not having subclinical mastitis.

These findings thus provide additional evidence that supplementation of one or more of such nutrients in the diet can prevent and/or treat subclinical mastitis.

Based on those unexpected findings, the inventors have developed and refined the inventions: by re-establishing an adequate level of the selected nutrient/mineral and at the same time by providing a known effector probiotic able to modulate the occurrence of mastitis, the inventors synergistically potentialize the body response in order to treat and prevent mastitis.

TABLE 1

| | Median intakes of | | | |
|---|---|---|---|---|
| | energy and nutrients | | | Difference in |
| | Women | Women | | median |
| | with | with | | intakes |
| | SCM* at | SCM at | wilcoxon- | between |
| | any visit | no visit | test | SCM and |
| | n = 52 | n = 125 | p value | no SCM |
| Fibre, g | 16 | 21 | 0.00 | −4.495 |
| Potassium, mg | 2773 | 3104 | 0.01 | −330.971 |
| Magnesium, mg | 265 | 313 | 0.01 | −48.003 |
| Phosphorus, mg | 1254 | 1340 | 0.01 | −85.926 |
| Calcium, mg | 744 | 974 | 0.00 | −229.719 |
| Manganese, mg | 2 | 3 | 0.02 | −0.498 |
| Iron, total, mg | 9 | 11 | 0.06 | −1.632 |
| Copper, mg | 1.2 | 1.4 | 0.01 | −0.213 |
| Beta-carotene, μg | 2068 | 2533 | 0.03 | −464.783 |
| Vitamin E, mg | 8 | 10 | 0.02 | −1.467 |
| Vitamin C, mg | 95 | 113 | 0.01 | −18.370 |
| Folate, ug | 263 | 309 | 0.01 | −46.740 |

TABLE 2

| | Women with | Women with | Adjusted** | |
|---|---|---|---|---|
| | SCM* at any visit | SCM at no visit | difference in | |
| | (Adjusted** | (Adjusted** | geometric mean | wilcoxon- |
| Average Diet | geometric | geometric | intakes between | test |
| of V2-V3 | means | means | SCM and no SCM | p value |
| Fibre, g | 17.5 | 21.6 | −4.16 | 0.00 |
| Potassium, mg | 2740.1 | 3225.5 | −485.40 | 0.00 |
| Beta-carotene, μg | 1791.9 | 2468.3 | −676.48 | 0.01 |
| Vitamin C, mg | 98.5 | 128.1 | −29.59 | 0.02 |
| Thiamin (vitamin | 1.5 | 1.8 | −0.29 | 0.04 |
| B1), mg | | | | |
| Riboflavin | 1.7 | 2.0 | −0.31 | 0.02 |
| (Vitamin B2), mg | | | | |
| Panthotenic acid | 5.2 | 6.0 | −0.86 | 0.02 |
| (D-panthotenate, | | | | |
| vitamin B5), mg | | | | |
| Vitamin B6 | 1.8 | 2.2 | −0.36 | 0.01 |
| (total), mg | | | | |
| Folate, total, μg | 305.4 | 372.0 | −66.61 | 0.01 |
| Vitamin B12 | 4.67 | 5.97 | −1.30 | 0.01 |
| (cobalamine), μg | | | | |
| Zinc, mg | 9.46 | 10.84 | 1.39 | −0.0325 |
| Selenium, μg | 130.96 | 156.64 | 25.68 | −0.0064 |

Women with SCM are defined as those with human milk sodium potassium (Na/K) ratio >0.6 during any of the following visits: days 2, 17, and 30), those with no SCM are defined as having a Na/K ratio ≤1.6 during any of the following visits: days 2, 17, and 30).
   adjusted for: country, mode of delivery

### Reference Example 2

Methods

Within the framework of a multicentre European observational study to characterise the human milk (HM) composition in the first four months of lactation (Atlas of Human Milk Nutrients study), we set out to understand whether there are differences in the HM composition between lactating women with subclinical mastitis (SCM) versus those without based on the sodium/potassium (Na/K) ratios in the HM.

Study Protocol

The ATLAS study was conducted in seven countries across Europe (France, Italy, Norway, Portugal, Romania, Spain and Sweden) as a longitudinal, observational, cohort in which HM as well as multiple maternal and infant parameters were collected at six time points post-partum (0-3 d, 17±3 d, 30±3 d, 60±5 d, 90±5 d and 120±5 d). Institutional and local Ethical boards of each centre approved the study. The participants provided a written informed consent form to participate in the study after receiving explanations and having read and understood the purpose and the objective of the study in their respective local languages. Pregnant women were recruited before delivery, generally during the last trimester of pregnancy. Inclusion criterial for this study were: (a) pregnant women between ages of 18 and 40 years; (b) BMI between 19 and 29, inclusive; (c) intention to breastfeed at least until 4 months post-partum; and (d) agreement to the study protocol and signed informed consent form. Exclusion criteria for this study were: (a) currently participating in another trial; (b) presenting conditions that contraindicate breastfeeding; (c) medical conditions or on medications for conditions such as metabolic and cardiovascular abnormalities; (d) dietary probes such as anorexia or bulimia; and (e) subjects not able to comply to the study procedures. Dedicated, trained and certified research nurses and assistants collected all data for this study. Maternal data included: demography, anthropometry, medical history, history of dietary supplements and three-day food diaries. Infant data included: demography, anthropometry, history of medication use, body composition (one centre in France and one in Sweden) and infant intake diary (three centres in France only).

Standardised Human Milk Sampling

HM sampling was standardised for all subjects. Milk was collected at 11 h00±2 h00 using an electric breast pump (Medela Symphony). For each mother, milk was collected from the same breast for the entire study and mothers were requested to empty the breast in the previous feed. This collected single full breast milk samples were mixed and an aliquot of 10-40 mL HM for each time point was collected. For colostrum, or the first time point 5-10 mL was collected. The remainder of the HM was returned to the mother for feeding to the infant at a later time point, if so required. Each collected HM sample was transferred to freezing tubes, labelled with subject number and collection information, stored at −18° C. in the home freezer, transferred to the hospital for storage at −80° C. and then shipped on dry ice to the Nestle Research Centre (Lausanne, Switzerland) where it was stored at −80° C. until analysis. The frozen HM samples were thawed once for aliquoting into 15 individual small volume fractions (0.2 mL to 2 mL) in separate polypropylene tubes dedicated to the different analyses.

Assessment of SCM Status

Lactating women were categorised in to two groups: those having any SCM (defined as Na/K ratio >0.6) and those normal (defined as Na/K ratio 0.6) based on the Na/K ratios in HM in early lactation (days 2, 17 and 30). Lactating women having at least 1 instance of SCM during any of these three time points were classified as having any SCM, while those in the normal category did not have any instance of SCM in any of these time points.

Fatty Acid Quantification in HM

Fatty acid profiles were determined by preparing the methyl esters of fatty acids (FAMEs). A direct transesterification of HM was performed with methanolic chloridric acid solution as described by Cruz-Hernandez et al. (Cruz-Hernandez, C. et al. (2017) J Sep Sci 40: 3289-3300). Briefly, into a 10 mL screw cap glass test tube, milk (250 μL) was added and mixed with 300 μL of internal standard FAME 11:0 solution (3 mg/mL) and 300 μL of internal standard TAG 13:0 solution (3 mg/mL). After addition of 2 mL of methanol, 2 mL of methanolic chloridric acid (3 N) and 1 mL of hexane, the tubes were heated at 100° C. for 90 min. To stop the reaction 2 mL of water was added and after centrifugation (1200 g×5 min) the upper phase (hexane) was transferred into gas chromatography vials. The analysis of FAMEs was performed by GC using a CP-Sil 88 capillary column (100 m, 0.25 mm id. 0.25 μm film thickness) and their identification by comparison of retention time with authentic standards (GC standard Nestlé 36 from NuCheck-Prep, Elysan Minn. USA).

Protein Quantification in HM

Total protein content in HM was measured using the colorimetric bicinchoninic acid (BCA) method according to the protocol provided with the BCA assay kit (ThermoFisher Scientific). The four major HM proteins alpha-lactalbumin, lactoferrin, serum albumin and caseins were quantified using a LabChip system as described previously (Affolter et al. (2016) Nutrients 8: 504).

Mineral Quantification in HM

Quantification of minerals was realised using Inductively Coupled Plasma Mass Spectrometry (ICP-MS).

For Sodium (Na), Magnesium (Mg), Phosphorous (P), Potassium (K), Calcium (Ca), Manganese (Mn), Iron (Fe), Copper (Cu), Zinc (Zn) and Selenium (Se), 0.7 mL of human breast milk was transferred into PFA vessels and mineralised in a CEM® Microwave digestion system using HNO₃/H₂O₂. Mineralised samples were transferred to PE tubes, diluted with MQ water and Germanium (Ge) and Tellurium (Te) were added as internal standards. Quantification was realised by ICP-MS using He as collision gas.

Certified Reference Materials (CRM) were added to all analytical series to control the quality of the quantification.

Results

The concentrations of iron, manganese, magnesium, copper, zinc, selenium, calcium, phosphorous, DHA, 18:3 n-3 octadecatrienoic acid, alpha-lactalbumin, lactoferrin and albumin in both the milk of mothers with sub-clinical mastitis and the milk of normal mothers at the 6 time-points post-partum are shown in Tables 2-4.

Women with sub-clinical mastitis have higher concentrations of iron, manganese, magnesium, copper, zinc and selenium; and lower concentrations of calcium and phosphorous in their milk in comparison to normal women.

The n-3 fatty acids docosahexaenoic acid (DHA) and 18:3 n-3 octadecatrienoic acid (alpha-linolenic acid) are present at lower concentrations in the milk of women with sub-clinical mastitis in comparison to normal women.

In addition, alpha-lactalbumin, lactoferrin and albumin are present at higher concentrations in the milk of women with sub-clinical mastitis in comparison to normal women.

The minerals that exhibit lower concentrations in the milk of women with sub-clinical mastitis (e.g. calcium and phosphorous) correlate with deficiencies that may be causing or contributing to the sub-clinical mastitis. Supplementation with such minerals may therefore prevent or treat the sub-clinical mastitis. In addition, the minerals with higher concentrations in the milk of women with sub-clinical mastitis (e.g. iron, manganese, magnesium, copper, zinc and selenium) correlate with the natural use of such minerals in countering infection and/or inflammation. Supplementation with such minerals may therefore be beneficial to the natural fight against infection and inflammation, thereby preventing or treating the sub-clinical mastitis.

Without wishing to be bound by theory, this rationale is supported by the knowledge that selenium improves antibacterial activity in milk and that selenium supplementation improves symptoms associated with mastitis in cows; similarly, copper and zinc have also been shown to reduce mastitis symptoms in cows and to enhance the immune system (O'Rourke, D. (2009) Irish Veterinary Journal 62 Supplement: 15-20).

It is believed that the elevated mineral concentrations observed in their data may result from increased uptake or hyper-accumulation from serum as part of host defence mechanisms to combat inflammation, which is consistent with roles for, for example, iron manganese and magnesium in immune function and countering inflammation (Rahmani, S. et al. (2015) J Nutr Food Sci 5: 1; Son, E. W. et al. (2007) Arch Pharm Res 30: 743-749; Maggini, S. et al. (2007) Br J Nutr 98 Suppl 1: S29-35; Tam, M. et al. (2003) Eur J Clin Nutr 57: 1193-1197; Kim, D. J. et al. (2010) Diabetes Care 33: 2604-2610; King, D. E. et al. (2005) J Am Coll Nutr 24: 166-171; Song, Y. et al. (2007) Am J Clin Nutr 85: 1068-1074).

The inflammatory state associated with sub-clinical mastitis alters the levels and ratios of fatty acids in milk. In particular, it has been found that fatty acid concentrations vary in the milk of women with sub-clinical mastitis. For example, it was found that the n-3 fatty acids docosahexaenoic acid (DHA) and 18:3 n-3 octadecatrienoic acid (alpha-linolenic acid) are present at lower concentrations in the milk of women with sub-clinical mastitis in comparison to normal women. It was also found higher n-6:n-3 ratios and higher arachidonic acid (ARA):DHA ratios in the milk of women with sub-clinical mastitis in comparison to normal women.

The higher n-6:n 3 ratio, ARA:DHA ratio and lower amounts of DHA all point towards a pro-inflammatory state. Supplementation with n-3 fatty acids, such as DHA and alpha-linolenic acid, may therefore also be used in treating or preventing the sub-clinical mastitis in a similar manner to that disclosed herein with respect to minerals such as calcium and phosphorous.

In addition, the inventors it was found that alpha-lactalbumin, lactoferrin and albumin are present at higher concentrations in the milk of women with sub-clinical mastitis in comparison to normal women. Supplementation with these proteins may therefore also be used in treating or preventing the sub-clinical mastitis in a similar manner to that disclosed herein with respect to minerals such as iron, manganese, magnesium, copper, zinc and selenium.

TABLE 2
Mineral and trace element concentrations of human milk across lactation
by presence or absence of sub-clinical mastitis (SCM).

| | | Day | Day | Day | Day | Day | Day | Overall |
|---|---|---|---|---|---|---|---|---|
| | | 2 | 17 | 30 | 60 | 90 | 120 | P value |
| Iron | Any SCM | 601 ± 392 | 461 ± 310 | 346 ± 162 | 292 ± 141 | 264 ± 140 | 216 ± 103 | <0.0001 |
| (μg/L) | | (n = 91) | (n = 97) | (n = 84) | (n = 78) | (n = 74) | (n = 71) | |
| | Normal | 415 ± 193 | 389 ± 186 | 326 ± 137 | 278 ± 132 | 245 ± 127 | 262 ± 502 | |
| | | (n = 111) | (n = 192) | (n = 174) | (n = 166) | (n = 156) | (n = 148) | |
| Manganese | Any SCM | 6.6 ± 3.9 | 4.4 ± 1.6 | 3.7 ± 1.1 | 4.5 ± 3.4 | 3.4 ± 0.8 | 3.7 ± 0.9 | <0.0001 |
| (μg/L) | | (n = 79) | (n = 41) | (n = 26) | (n = 22) | (n = 19) | (n = 13) | |
| | Normal | 5.8 ± 3.1 | 3.9 ± 1.3 | 3.5 ± 1.0 | 3.4 ± 0.9 | 3.3 ± 0.7 | 4.0 ± 2.2 | |
| | | (n = 92) | (n = 103) | (n = 75) | (n = 45) | (n = 36) | (n = 37) | |
| Magnesium | Any SCM | 38.5 ± 8.5 | 35.0 ± 6.2 | 34.2 ± 5.5 | 37.4 ± 5.8 | 38.8 ± 5.4 | 38.4 ± 5.4 | 0.076 |
| (mg/L) | | (n = 87) | (n = 94) | (n = 80) | (n = 76) | (n = 72) | (n = 69) | |
| | Normal | 38.0 ± 6.5 | 34.3 ± 5.7 | 33.9 ± 5.2 | 36.5 ± 5.6 | 38.6 ± 5.9 | 39.3 ± 6.2 | |
| | | (n = 106) | (n = 171) | (n = 149) | (n = 162) | (n = 154) | (n = 145) | |
| Copper | Any SCM | 533 ± 267 | 529 ± 135 | 423 ± 86 | 325 ± 74 | 268 ± 72 | 221 ± 69 | <0.001 |
| (μg/L) | | (n = 91) | (n = 97) | (n = 84) | (n = 78) | (n = 74) | (n = 71) | |
| | Normal | 472 ± 175 | 522 ± 111 | 422 ± 89 | 304 ± 76 | 256 ± 77 | 228 ± 89 | |
| | | (n = 111) | (n = 192) | (n = 174) | (n = 166) | (n = 156) | (n = 148) | |
| Zinc | Any SCM | 7975 ± 3093 | 3234 ± 1274 | 2422 ± 906 | 1616 ± 1208 | 1113 ± 521 | 996 ± 520 | <0.0001 |
| (μg/L) | | (n = 91) | (n = 97) | (n = 84) | (n = 78) | (n = 74) | (n = 71) | |
| | Normal | 7178 ± 2734 | 3590 ± 1051 | 2686 ± 872 | 1599 ± 692 | 1219 ± 571 | 1031 ± 563 | |
| | | (n = 111) | (n = 192) | (n = 174) | (n = 166) | (n = 153) | (n = 148) | |
| Selenium | Any SCM | 37 ± 19 | 19 ± 6 | 15 ± 3 | 12 ± 3 | 10 ± 2 | 9 ± 2 | <0.0001 |
| (μg/L) | | (n = 91) | (n = 97) | (n = 84) | (n = 78) | (n = 74) | (n = 71) | |
| | Normal | 26 ± 12 | 18 ± 3 | 15 ± 3 | 12 ± 2 | 10 ± 2 | 10 ± 6 | |
| | | (n = 111) | (n = 192) | (n = 174) | (n = 166) | (n = 156) | (n = 148) | |
| Calcium | Any SCM | 256 ± 73 | 281 ± 54 | 296 ± 47 | 299 ± 41 | 288 ± 45 | 271 ± 36 | 0.003 |
| (mg/L) | | (n = 91) | (n = 97) | (n = 84) | (n = 78) | (n = 74) | (n = 71) | |
| | Normal | 295 ± 72 | 293 ± 55 | 296 ± 51 | 301 ± 45 | 297 ± 48 | 284 ± 42 | |
| | | (n = 111) | (n = 192) | (n = 174) | (n = 166) | (n = 156) | (n = 148) | |
| Phosphorus | Any SCM | 109 ± 41 | 150 ± 31 | 149 ± 29 | 138 ± 21 | 128 ± 18 | 126 ± 20 | <0.0001 |
| (mg/L) | | (n = 91) | (n = 97) | (n = 84) | (n = 78) | (n = 74) | (n = 71) | |
| | Normal | 144 ± 32 | 169 ± 28 | 155 ± 24 | 139 ± 21 | 133 ± 20 | 131 ± 22 | |
| | | (n = 111) | (n = 192) | (n = 174) | (n = 166) | (n = 156) | (n = 148) | |

TABLE 3
Fatty acid concentrations of human milk across lactation by presence or absence of sub-clinical mastitis (SCM).

| | | Day | Day | Day | Day | Day | Day | Overall |
|---|---|---|---|---|---|---|---|---|
| | | 2 | 17 | 30 | 60 | 90 | 120 | P value |
| DHA | Any SCM | 11.5 ± 7.8 | 12.6 ± 9.0 | 10.5 ± 5.8 | 10.3 ± 6.1 | 9.5 ± 10.0 | 8.4 ± 5.9 | 0.0001 |
| (mg/100 mL) | | (n = 103) | (n = 103) | (n = 89) | (n = 78) | (n = 77) | (n = 74) | |
| | Normal | 13.7 ± 8.8 | 15.1 ± 10.2 | 13.6 ± 11.0 | 11.3 ± 8.4 | 10.8 ± 6.9 | 11.7 ± 15.8 | |
| | | (n = 158) | (n = 187) | (n = 168) | (n = 155) | (n = 144) | (n = 137) | |
| 18:3 n-3 | Any SCM | 13.9 ± 11.2 | 21.8 ± 19.4 | 24.5 ± 18.7 | 27.8 ± 24.7 | 27.3 ± 27.6 | 23.6 ± 18.8 | <0.0001 |
| octadecatrienoic | | (n = 100) | (n = 104) | (n = 90) | (n = 80) | (n = 77) | (n = 76) | |
| acid | Normal | 17.0 ± 11.3 | 29.1 ± 21.9 | 29.3 ± 23.7 | 32.8 ± 33.8 | 27.9 ± 24.7 | 32.3 ± 30.4 | |
| (mg/100 mL) | | (n = 159) | (n = 187) | (n = 171) | (n = 161) | (n = 154) | (n = 144) | |

TABLE 4
Protein concentrations of human milk across lactation by presence or absence of sub-clinical mastitis (SCM).

| | | Day | Day | Day | Day | Day | Day | Overall |
|---|---|---|---|---|---|---|---|---|
| | | 2 | 17 | 30 | 60 | 90 | 120 | P value |
| Alpha- | Any SCM | 3232.8 ± 685.0 | 3131.2 ± 548.4 | 2893.1 ± 506.6 | 2469.0 ± 466.5 | 2249.5 ± 422.3 | 2089.4 ± 415.8 | <0.0001 |
| lactalbumin | | (n = 102) | (n = 104) | (n = 91) | (n = 81) | (n = 77) | (n = 75) | |
| (ng/μL) | Normal | 3043.6 ± 597.1 | 3036.6 ± 544.4 | 2740.7 ± 499.1 | 2278.1 ± 372.4 | 2126.7 ± 374.1 | 2015.8 ± 378.0 | |
| | | (n = 128) | (n = 187) | (n = 170) | (n = 162) | (n = 156) | (n = 147) | |
| Lactoferrin | Any SCM | 7053.9 ± 3832.6 | 3315.6 ± 2486.1 | 2430.8 ± 2176.9 | 1757.1 ± 955.4 | 1543.1 ± 699.2 | 1510.4 ± 1502.3 | <0.0001 |
| (ng/μL) | | (n = 102) | (n = 104) | (n = 91) | (n = 81) | (n = 77) | (n = 75) | |
| | Normal | 5340.2 ± 2372.7 | 2379.4 ± 913.4 | 1765.9 ± 680.7 | 1339.0 ± 485.6 | 1298.6 ± 810.1 | 1468.2 ± 3269.1 | |
| | | (n = 128) | (n = 187) | (n = 170) | (n = 162) | (n = 156) | (n = 147) | |
| Albumin | Any SCM | 1534.6 ± 1821.9 | 729.2 ± 671.1 | 659.1 ± 655.3 | 536.5 ± 190.5 | 474.7 ± 130.2 | 483.8 ± 388.8 | <0.0001 |
| (ng/μL) | | (n = 102) | (n = 104) | (n = 91) | (n = 81) | (n = 77) | (n = 75) | |
| | Normal | 813.3 ± 1342.5 | 551.8 ± 160.6 | 529.7 ± 301.8 | 469.2 ± 123.3 | 455.8 ± 126.8 | 516.8 ± 894.1 | |
| | | (n = 128) | (n = 187) | (n = 170) | (n = 162) | (n = 156) | (n = 147) | |

Further example: The mix of the invention or the composition of the invention can, for example, be a commercially available, conventional, nutritional composition for pregnant women, to which the selected probiotic L. fermentum CECT-5716 and the selected nutrient/mineral are incorporated.

All publications mentioned in the above specification are herein incorporated by reference. Various modifications and variations of the disclosed agents, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for use in treating or preventing mastitis in a subject comprising administering a mix comprising a probiotic Lactobacillus Fermentum CECT-5716 and a nutrient selected from the group consisting of: beta-carotene, fiber, Vitamin C, Folate, Vitamin B1, Vitamin B2, Vitamin B5, Vitamin B6, Vitamin B12 or Potassium and a combination of two of more thereof to the subject.

2. The method according to claim 1 which is a combination of nutrients comprising fiber and folate.

3. The method according to claim 1 which is a combination of nutrients comprising fiber and one vitamin selected from the group consisting of: Vitamin B1, Vitamin B2, Vitamin B5, Vitamin B6 and Vitamin B12.

4. The method according to claim 1 which is a combination of nutrients comprising folate and one vitamin selected from the group consisting of: Vitamin B1, Vitamin B2, Vitamin B5, Vitamin B6 and Vitamin B12.

5. The method according to claim 1, which is a combination of nutrients comprising fiber, folate and one vitamin selected from the group consisting of: Vitamin B1, Vitamin B2, Vitamin B5, Vitamin B6 and Vitamin B12.

6. The method according to claim 1, wherein the nutrient is in combination with one or more further nutrients selected from the group consisting of beta-carotene, fiber, Vitamin C, Folate, Vitamin B1, Vitamin B2, Vitamin B5, Vitamin B6, Vitamin B12 and Potassium.

7. The method according to claim 1, wherein the nutrient is in combination with vitamin E.

8. The method according to claim 1, wherein the combination further comprises a mineral selected from the group consisting of: iron, manganese, magnesium, copper, zinc and selenium.

9. The method according to claim 1, wherein the nutrient is in combination with:
(a) an n-3 fatty acid, preferably wherein the nutrient is in combination with a fatty acid selected from the group consisting of docosahexaenoic acid (DHA) and 18:3 n-3 octadecatrienoic acid (alpha-linolenic acid);
(b) a protein selected from the group consisting of alpha-lactalbumin, lactoferrin and albumin; and/or
phosphatidylcholine and/or lecithin.

10. The method according to claim 1, wherein the mix is in the form of a nutritional composition.

11. The method according to claim 1, wherein the mastitis is sub-clinical mastitis or clinical mastitis, preferably sub-clinical mastitis.

12. The method according to claim 1, wherein the subject is at risk of suffering from sub-clinical mastitis or clinical mastitis.

13. The method according to claim 1, wherein the treatment or prevention increases the probability of initiating and/or continuing breastfeeding by the subject; and/or increases the probability of the subject exclusively breast-feeding her infant and/or increases the duration of breastfeeding of the subject.

14. The method according to claim 1, wherein the subject continues to breast-feed for at least 4 months.

15. The method according to claim 1, wherein the treatment or prevention increases the quality and/or quantity of the subject's breast milk.

16. The method according to claim 1 in a subject who is a lactating woman.

17-18. (canceled)
